(19) United States
(12) Patent Application Publication
(10) Pub. No.: US 2006/0008843 A1
(43) Pub. Date: Jan. 12, 2006

(54) Determination of protein function

(75) Inventors: Kris F. Sachesenmeier, Pittsburgh, PA (US); Douglas Koebler, Irwin, PA (US); Darrin Sabol, Pittsburgh, PA (US); Alfred B. Bahnson, Pittsburgh, PA (US); Raymond K. Houck, Oakmont, PA (US)

Correspondence Address: FOLEY AND LARDNER LLP;SUITE 500, 3000 K STREET NW, WASHINGTON, DC, 20007, US

(73) Assignee: Automated Cell, Inc

(21) Appl. No.: 10/514,197

(22) PCT Filed: May 13, 2003
(86) PCT No.: PCT/US03/14743

§ 371 Date: Apr. 26, 2005

(30) Foreign Application Priority Data
May 17, 2002 (US) 60381089

(51) Int. Cl. G01N 33/53; G06F 19/00

(52) U.S. Cl. 435/7.1; 702/19

(57) ABSTRACT

For purposes of determining the function of a protein, an automated system captures images of cells, each cell located in a predetermined well. After a given cell is exposed to a protein of interest, the system measures the responses of the cell over time, evaluating a variety of cellular parameters. Analytical software within the system evaluates data generated by these measurements, at single-cell resolution. By comparing with various controls the data thus obtained, the system illuminates the function of a protein with respect to one or more disease models, independent of information regarding the structure, chemistry or underlying genomics of the protein.

### FIELD OF INVENTION

The present invention relates to the field of proteomics, which encompasses the study of the expression, modification, interactions and function of proteins. More specifically, this invention relates to functional proteomics, which focuses on how proteins function in the human body and how they impact human health and disease.

### BACKGROUND OF THE INVENTION

Proteins are involved in every biological function. They affect biological processes directly, such as through protein signal transduction, and indirectly, such as by enzymes and hormones. Proteins also are involved in disease responses and progressions, such as the inflammatory response to an injury, and the deadly course that malignant tumors take if left unchecked. Proteins determine the shape, structure, division, growth, behavior and death of cells. Proteins are the main instruments of molecular recognition and catalysis, participating in every cellular process and reaction.

Proteins are made from an assortment of 20 amino acids strung together like pearls on a necklace. The DNA comprising a protein's gene determines the type and order of amino acids in a protein. The human genome comprises approximately 35,000 genes. These genes produce approximately 300,000 to 500,000 proteins. The specific sequence of amino acids dictates a protein's structure, called its conformation. The precise chemical properties of a protein's conformation enable the protein to perform a specific catalytic or structural function in a cell. Thus, the structure of a protein is a strong determinant of its function. In fact, proteins with similar or related structures often imply related functions.

While the nucleotide sequences of genes that make up the human genome recently have been elucidated, the function has been determined for only about 20% to 30% of the encoded proteins. Since establishing protein function is a key part of any drug discovery effort, drug companies have employed a variety of methods to infer protein function. For example, researchers often infer protein function by comparison to homologous proteins that have established functions. One such method uses mass spectrometry to define the linear sequence of amino acids that make up a protein molecule. Computer models then are employed to compare the composition and conformation of a protein of interest to those of known proteins. Based on the observed homology, the protein is assigned a putative function.

Researchers also examine protein-protein associations to infer disease-linked function. Mass spectrometry can be used to investigate protein-protein interactions by the isolating protein complexes and subsequently identifying the proteins in the complexes. Yeast two-hybrid systems also have been developed to study protein interactions as described, for example, in U.S. Pat. No. 6,057,101. These systems evaluate protein-protein interactions by isolating proteins that interact with the protein of interest, typically by screening a cDNA library.

Another method-for studying protein-protein interaction is phage display. The basic process is to grow and select bacteriophages that express certain antibodies or proteins at their surfaces. The resulting phages are evaluated to determine which phages bear antibodies with a high affinity for the selected antigen.

A variety of cell-based assays have been employed to evaluate protein-protein interactions. Examples include, but are not limited to, in vitro cytotoxity assays, soft agar colony formation assays, in vitro anti-microbial assays and assays that detect changes in cellular morphology of the cancer cells. Automated versions of these assays also have been developed. For example, see U.S. Pat. No. 6,127,133 and No. 6,232,083.

Disease-linked expression profiling also is employed to infer protein function. Two-dimension (2D) gel separation is an example of this method. The 2D gel method separates proteins in a sample by displacement in two dimensions. After isolation, the proteins are further studied or characterized, usually by mass spectrometry. The 2D gel method is further explained in patents U.S. Pat. No. 6,278,794 and No. 6,064,754. Existing 2D gel methods can identify proteins that are expressed differentially in diseased verses healthy tissue or cells. Identified proteins can then be analyzed by mass spectrometry to identify the specific protein composition.

Protein microarrays also can be used in disease-linked expression profiling. Typically a multiple-well plate or slide will contain many different combinations of proteins. This method can be used to study protein-protein interactions and protein-ligand interactions. Miniaturized assays are used to accommodate extremely low sample volumes and to enable the rapid, simultaneous processing of thousands of proteins.

While a variety of approaches are available to infer protein function, the methods are labor intensive, costly and typically generate both false positives and false negatives. Furthermore, the challenge of demonstrable functional relevance remains an inevitable downstream step in the development of promising drug candidates. Moreover, since a protein's putative function can often differ from its real function, the current practice of determining functional relevance during the later stages of development increases the cost and cycle time of drug discovery.

### SUMMARY OF THE INVENTION

Accordingly, the present invention addresses a need for an efficient and cost-effective approach to determining the function of a protein.

The invention also addresses a need for a methodology that correlates protein function to aspects of a pathology, independent of information about the structure or molecular biology of the protein.

In meeting these and other needs, there has been provided, in accordance with one aspect of the present invention, a protein-analysis method comprising (A) bringing a protein into contact with at least a first disease-model cell and a second disease-model cell, respectively, wherein each of the first and second cells is located in a separate well; then (B) determining the dynamic state of each of the cells, whereby a data set is generated for each cell; and (C) analyzing the data set for the first cell and the data set for the second cell, to obtain information about the function of the protein. In one embodiment, the data sets of step (C) address a plurality of cell parameters. The determination of the dynamic state can comprise imaging each of the cells by either visible or fluorescent light, or both. In another embodiment, the first disease-model cell and the second disease-model cell relate to the same disease model. In another aspect of the invention, the method further comprises providing a plurality of proteins, wherein step (A) comprises bringing into contact, with N number of disease-model cells, a chosen protein from the plurality such that each of at least some of the N cells contacts a different protein from the plurality, N being an integer greater than 2. The data sets of step (C) of such a method can address a plurality of M cell parameters, M being an integer of 1 or greater, and can be organized as an N x M array of values. In a preferred embodiment, the cell parameters comprise two or more of the measured parameters enumerated in Table I. In one aspect of the invention, more than one well receives the same protein from the plurality of proteins, while in another at least one well receives more than one protein from the plurality.

In preferred applications of the inventive method, either the first disease-model cell or the second disease-model cell employs an oncogenesis disease model, a primary immune response disease model or an angiogenesis disease model.

In other aspects of the invention, step (A) comprises bringing the protein into contact with a first plurality of first disease-model cells and a second plurality of second disease-model cells, respectively, and wherein the information distinguishes a subpopulation of at least one of the first and second pluralities.

In another embodiment, the present invention provides an integral array of biochambers, each (i) comprising a well in which a disease-model cell is located and (ii) defining a separate, closed environment for the cell, wherein each well contains a protein and the array presents a predetermined pattern of association between wells and proteins.

The invention also provides a protein-analysis method comprising (A) disposing a first disease-model cell in a first well in a manner wherein at least one cell is individually observable; (B) disposing a second disease-model cell in a second well in a manner wherein at least one cell is individually observable; (C) bringing a protein into contact with the first and second disease-model cells; (D) repeatedly observing the first and second disease-model cells; (E) compiling data in the form of data: sets pertaining to a change in at least one of a plurality of observable characteristics of each of the respective first and second disease-model cells, prior to and subsequent to the protein being contacted with the first and second disease-model cells; and (F) analyzing the data sets to obtain information about the function of the protein. In one embodiment, steps (A) through (D) are implemented robotically within a closed environment, while in another the step of repeatedly observing is carried out optically, without fixation of cells. In another embodiment, steps (A) through (F) are implemented robotically. Observable characteristics typically employed in the claimed method include, for example, cell movement, cell division, apoptosis, morphology, adherence and physiological function, as well as the measured parameters enumerated in Table I. In another embodiment, the method further comprises means for selectively adding a modifying agent in addition to the protein.

In another embodiment, the invention provides a protein-analysis apparatus comprising means for disposing a plurality of first disease-model cells in a first well in a manner wherein at least one of the first disease-model cells is individually observable; means for disposing a plurality of second disease-model cells in a second well a manner wherein at least one of the second disease-model cells is individually observable; means for bringing a protein into contact with at least one of the first and second disease-model cells; means for repeatedly observing the first and second disease-model cells; means for compiling and analyzing data in the form of data sets that pertain to a change in at least one of a plurality of observable characteristics of each of the respective first and second disease-model cells, prior to and subsequent to the protein being contacted with the first and second disease-model cells.

The invention further provides a protein-analysis method comprising (A) disposing a disease-model cell in a well in a manner wherein at least one cell is individually observable; (B) bringing a plurality of proteins into contact with the disease-model cell;(D) repeatedly observing the disease-model cell; (E) compiling data in the form of data sets pertaining to a change in at least one of a plurality of observable characteristics of disease-model cell, prior to and subsequent to the proteins being contacted with the disease-model cell; and (F) analyzing the data sets to obtain information about the function of the proteins. In one embodiment, the method further comprises isolating a protein of interest by splitting the plurality into a smaller number of pluralities and repeating steps (A) through (F), using the smaller number of pluralities for step (B). In another embodiment, the method further comprises isolating a protein of interest by splitting the plurality into individual proteins and repeating steps (A) through (F) for each of the proteins.

Other objects, features and advantages of the present invention will become apparent from the following detailed description. The detailed description and specific examples, while indicating preferred embodiments, are given for illustration only as various changes and modifications within the spirit and scope of the invention will become apparent to those skilled in the art.

### BRIEF DESCRIPTION OF THE DRAWINGS

FIG. 1 provides a schematic representation of the components of a device for carrying out the present invention.

FIGS. 2-5 present schematics of the chamber of the preferred device. FIG. 2 provides a front view of the biobox chamber on the moveable table. FIG. 3 presents a top view of the biobox chamber, while FIG. 4 provides a side view. FIG. 5 provides front, top and side views of the biobox off of the microscope.

FIG. 6 is a schematic representation of the pattern recognition software employed by the invention. Panel A presents modeled data representing a single cell one dividing cell, and two cells in contact, then two separated cells. Panel B presents the data derived from analysis of the objects in panel A.

FIG. 7 is an overhead view of a representation of the movement of the table to locate the sample well under the needle for fluid exchange with any point in the sample plate.

FIG. 8 is an overhead view of the movement of the table to locate the needle in the wash and waste station in the chamber.

FIG. 9 is a schematic representation of a z-robot pipette and fluidics elements.

FIG. 10 is a schematic representation of the z-robot pipette and fluidics elements on the biobox.

FIG. 11 provides a schematic of an exemplary data analysis procedure employed in the present invention.

FIG. 12 illustrates an evaluation of subpopulations of T lymphocytes. The left panel shows a single time image of the T lymphocytes. The Y-axis of the histogram in the right panel is the normalized population frequency, and the X-axis. is a fraction of the cells segregated by curvelinear velocity.

FIG. 13 provides a schematic of the oncogenesis disease model.

FIG. 14 depicts an example of the primary immune response disease model.

FIG. 15 provides a schematic of the angiogenesis disease model.

FIG. 16 presents the results from one assay (PIR-1) from a primary immune response disease model. Fluorescent images were superimposed upon visible light images (panels A and B) to align clusters of phagocytized beads with phagocytic dendritic cells (DCs). DCs were incubated for 24 hours with 2-micron fluorescent polystyrene beads in the presence (panel A) or absence (panel B) of IL-1 beta (20 ng/ml) and tumor necrosis factor (TNF). Cells containing fluorescent bead clusters of area greater than 60 square microns from duplicate wells are quantified in panel C.

FIG. 17 presents results from a second assay CPIR-2) from a primary immune response disease model. DCs were co-cultured with naive T cells for 24 hours and imaged every 3 minutes in the presence (panel B) or absence (panel A) of 1 ng/ml superantigen Staphylococcal Enterotoxin B. The number of T cells (TC) within a single T cell diameter (see arrows, no outlines) of a dendritic cell (DC) were quantified for each image and plotted per DC in panel C. T cells are outlined that were not located proximal to a dendritic cell.

FIG. 18 provides results from a third assay (PIR-3) from a primary immune response disease model. DCs were co-cultured for 24 hours with naive T cells (TC) in the presence (panel B) or absence (panel A) of Staphylococcus Enterotoxin B superantigen (1 ng/ml) and then imaged every 3 minutes. The ratio of cell length to breadth was calculated for every cell in each image. Panel C plots the image averages.

FIG. 19 provides results from a fourth assay (PIR-4) from a primary immune response disease model. Primary lymphocytes were isolated from peripheral blood and cultured in the presence (panel B) or absence (panel A) of IL-2, the protein of interest, at various concentrations (0.2, 1, 5, 25, and 100 ng/ml). Lymphocyte migration was quantified from single cell tracking and plotted over time (panel C).

FIG. 20 provides another example of a primary immune response model assay. DCs were cultured in the presence (panels B and D) or absence (panels A and C) of 50 ng/ml of TNF-alpha. The accumulated tracks for more than 300 images are shown by light lines. The average velocities for the cells over the period are plotted (Panel E), with error bars representing standard deviation.

FIG. 21 provides a 3-D graph showing that multiple assay determinations can be obtained from a single sample plate.

FIG. 22 depicts, in schematic form the operations of an exemplary software program useful for imaging cells in the present invention.

### DETAILED DESCRIPTION

The present invention allows for the direct determination of the function of a protein. An automated system captures images of cells in a well within a biochamber of a closed environment. After a given cell is exposed to a protein of interest, the system measures the dynamic state of cell, reflected in the responses of the cell over time to the protein, by evaluating a variety of cellular parameters, at single-cell resolution. Analytical software within the system evaluates data generated by these measurements. By comparing the kinetic data from the exposed cells with various controls, the system elucidates the function of a protein in one or more disease models.

The inventive method provides an efficient, cost-effective means for determining the function of a protein. In addition, protein function can be determined without knowing the structure, gene sequence or chemistry of the protein. Furthermore, the invention streamlines the development process and reduces the cost of drug discovery by elucidating the function of a target protein during the earliest stage of development. The invention can be used for screening, discovering, analyzing and validating disease and health relevance of proteins.

In one of its aspects, the present invention provides methodology and compositions for identifying lead developmental targets, in the form of proteins that have functions of interest. To this end, a plurality of proteins can be examined simultaneously by an automated system within the invention. Moreover, it is feasible to examine the effect of a combination of proteins on a particular cell type, as well as for a variety of disease models to be evaluated concomitantly.

In the present description, the terms “gene” and “structural gene” refer to a DNA sequence that is transcribed into messenger RNA (mRNA), which is then translated into a sequence of amino acids characteristic of a specific polypeptide (protein).

The term “expression” denotes the process by which a polypeptide is produced from a structural gene. The expression process involves transcription of the gene into MRNA and the translation of such rnRNA into polypeptide(s).

A “cloning vector” is a DNA molecule, such as a plasmid, cosmid, phagemid, or bacteriophage or other virally derived entity, that can replicate in a host cell and that is used to transform cells for gene manipulation. Cloning vectors typically contain one or more restriction endonuclease recognition sites at which foreign DNA sequences may be inserted in a determinable fashion without loss of an essential function of the vector, as well as a marker gene that is suitable for use in the identification and selection of cells transformed with the cloning vector. Appropriate marker genes typically include genes that provide various antibiotic or herbicide resistances. A variety of markers are available to the skilled artisan.

The phrase “disease-model cell” refers to one or more cells from a disease state of interest. A disease-model cell can comprise more than one type of cell. While they do not represent an exhaustive description of a disease state, disease-model cells provide an overview of the key events associated with a particular disease, which can be monitored to determine the function of a particular protein. Similarly, the disease-model cells can provide an overview of the key states of a healthy human without the particular disease of interest.

A “data set” is an assemblage of data generated for each cell regarding the various parameters measured during the experiment.

A “modifying agent” affects at least one of the plurality of observable characteristics of a disease-model cell.

In a preferred embodiment, a disease model is selected first. Then, the assays used to quantify different parts of the disease model are chosen. The assays incorporate the various primary cells, cell lines and engineered cells utilized by the disease model. Next, the protein library is selected. The library can consist of, for example, peptides, secreted proteins or antibodies. The library can take the form of isolated protein, such as those obtained using chromatography, 2D gel electrophoreses and protein chips, or DNA, such as a cDNA library. Next, the proteins (or CDNA) are added to the disease-model cells. The method of protein addition depends upon the specific form of the protein (or CDNA). If the protein is an antibody or protein supernate from a culture well, it can be added into a specific well by pipetting. If the protein needs to be delivered into the interior of the disease-model cells, then fusion protein methods, such as described in U.S. Pat. Nos. 5,804,604 and 5,747,641, or viral methods, such as found in U.S. Pat. Nos. 6,184,038 and 6,017,735, can be used. For cDNA, common transfection methods for incorporating cDNA sequences into cells can be used. After the proteins are added to the disease model, the functional assays are performed, and the quantitative data is collected using the imaging techniques described herein.

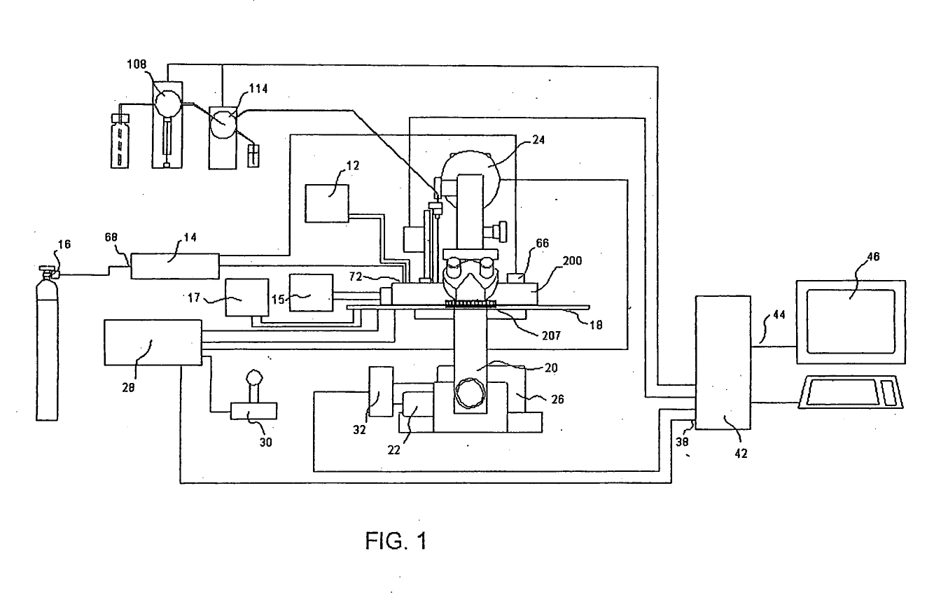

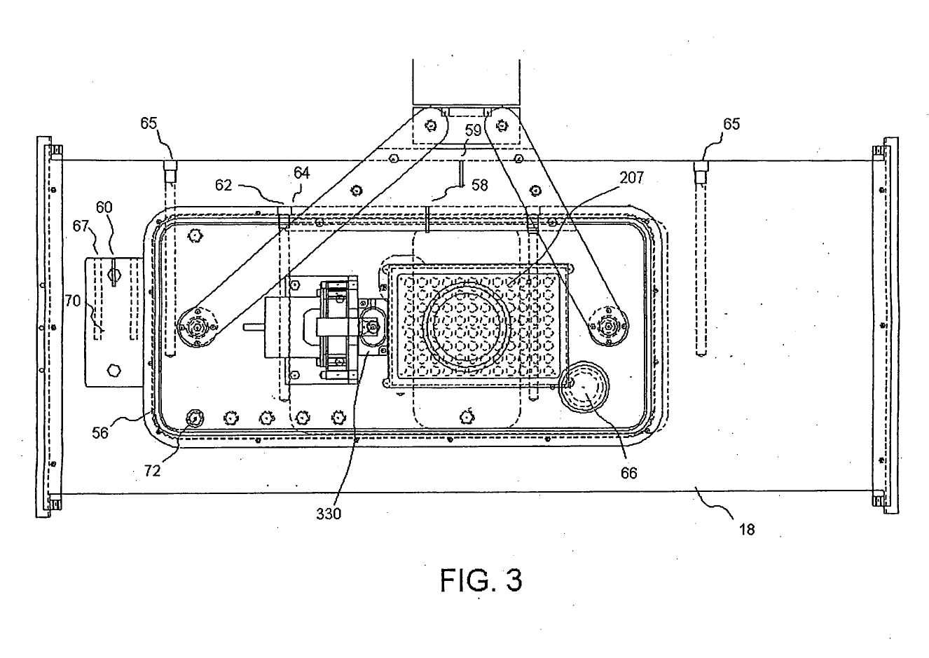

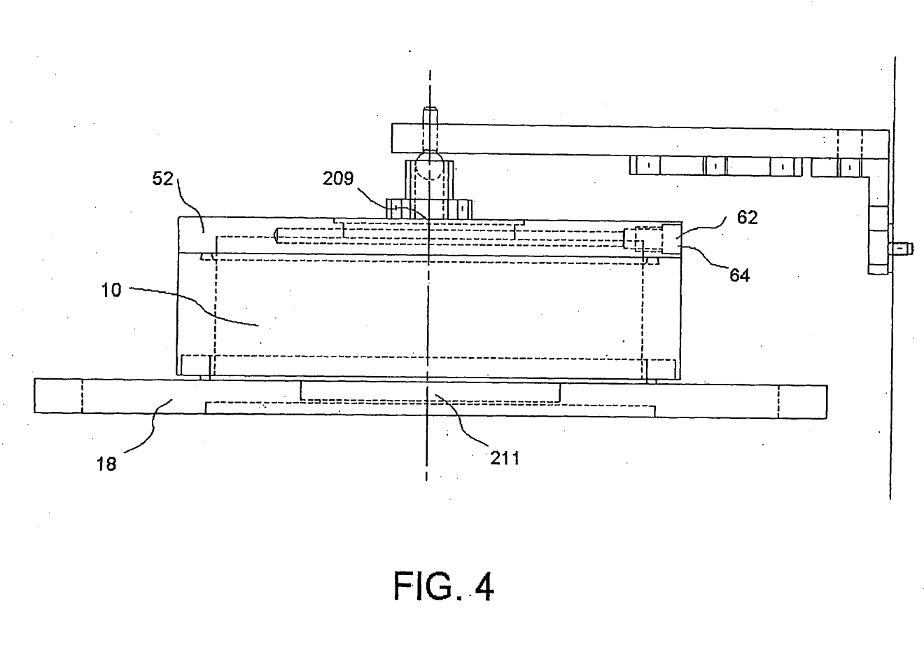

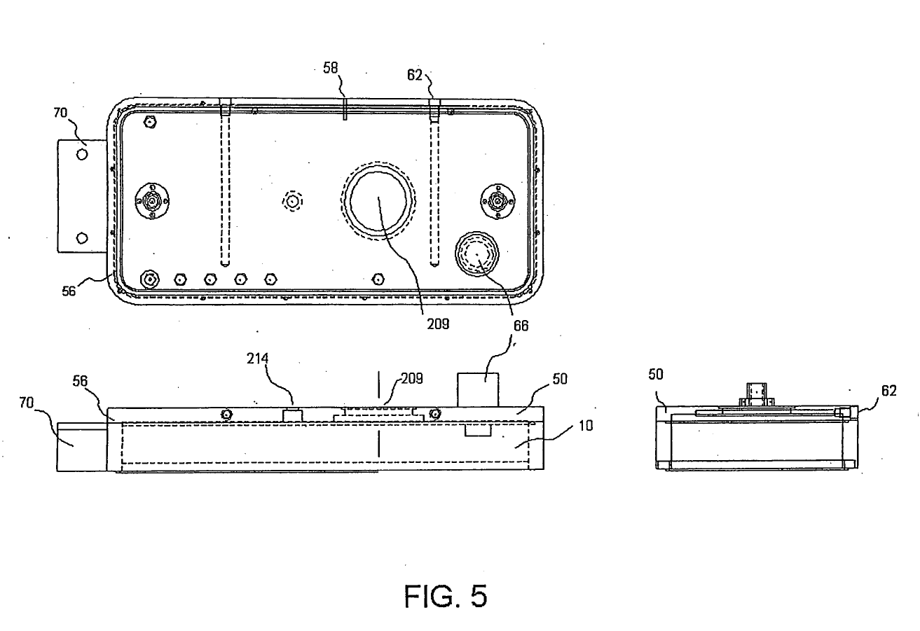

In a preferred embodiment, the methodology of the present invention is affected with the device described in U.S. Pat. No. 6,008,010, the contents of which are hereby incorporated by reference. As shown in FIGS. 1-5, the device includes an incubating mechanism 200, which preferably includes a housing 204 having a Biochamber 10 in the housing 204. The incubating mechanism 200 also preferably includes a first well 206 and at least a second well 208 in which cells are grown. The first and second wells are disposed in the Biochamber 10 of the housing 204. The incubating mechanism 200 preferably comprises a transparent plate 207 in which the first and second wells are disposed.

The housing 204 preferably has a first port mechanism 210 through which the first and second wells in the Biochamber 10 can be viewed. The first port mechanism 210 preferably includes a first window 209 disposed in the top of the housing 204 and a second window 211 disposed in the bottom of the housing 204 and in optical alignment with the first window 209 to form an optical path for light entering the first window 209 from outside the housing 204 and to exit the housing 204 through the second window 211. The housing 204 preferably has a second port mechanism 214 in fluid communication with the Biochamber 10.

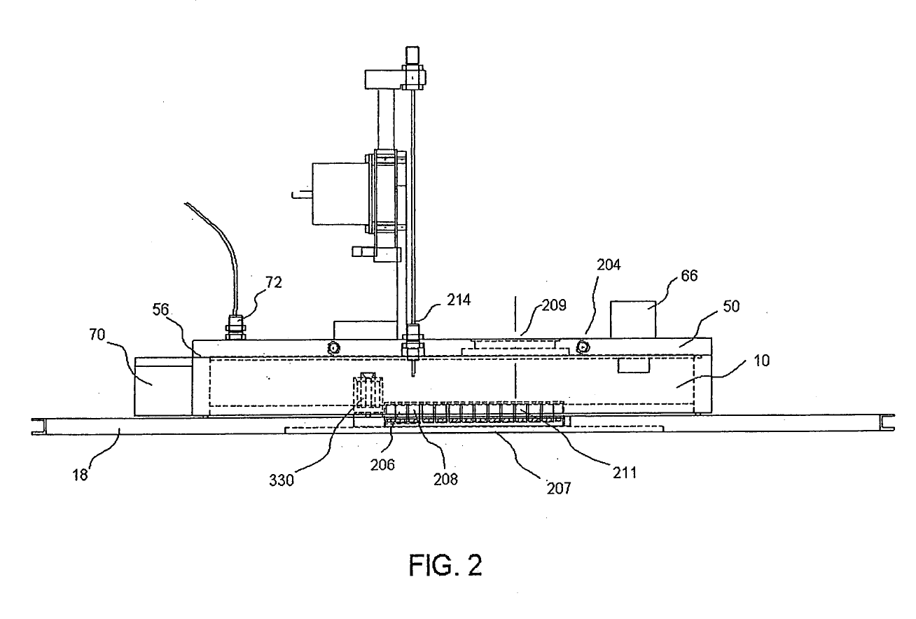

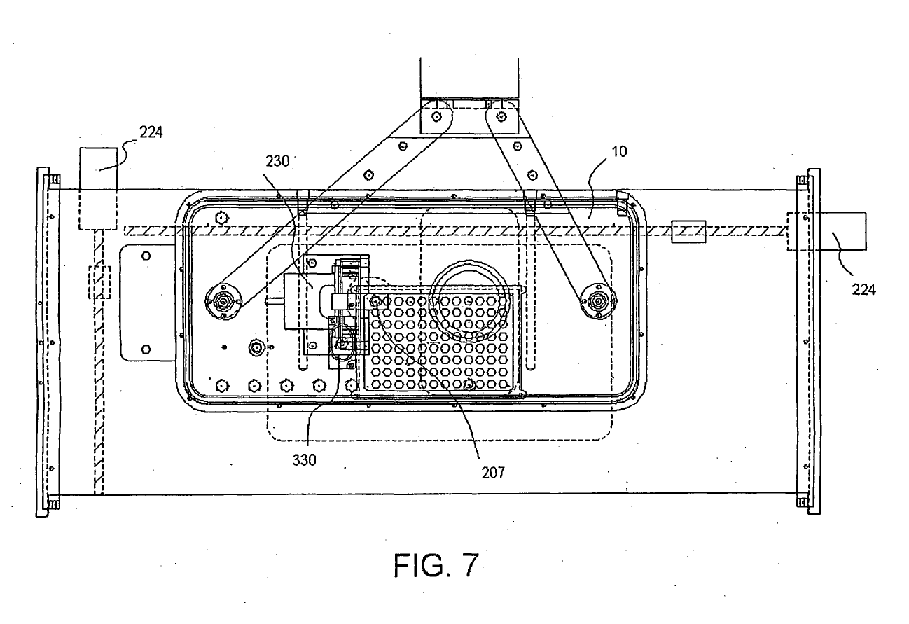

Cells are maintained in individual wells of multi-well plates under a sterile, controlled environment (i.e., physiological temperature, pH, pO₂ and humidity) inside an anodized aluminum Biochamber 10 with glass windows on top and bottom to provide an optical path for imaging. There are two embodiments for the system 300: a Biochamber 10 (FIG. 1 and Table II) and a Biochamber 10 also with z-robot for medium exchange, as shown in FIGS. 7-10. The Biochamber 10 for the first embodiment (described in detail in FIGS. 2-5 and Table III) is approximately 6 inches by 5 inches by 2 inches high. Temperature is regulated using and RTD 58, Temperature Controller 12, and Heating Cartridges 62. Media pH is maintained using standard bicarbonate-based buffers and a CO₂ Controller 14, which sets atmospheric pCO₂ at 5% by regulating the flow of CO₂ from a CO. Supply Tank with Regulator 16 through a solenoid valve, based on signals from a detachable CO₂ Sensor 66 mounted on the side of the Biochamber 10. Control of pO₂ in the Biochamber 10 can be maintained similarly through a sensor and supply interfaced through two additional chamber front ports. The humidity is maintained by a heated chamber 70 of sterile water to maintain close to 100% relative humidity inside the biobox and minimize evaporation.

In operation, before use the disassembled Biochamber 10 is sterilized by swabbing with a 70% aqueous solution of ethanol in the sterile environment of a laminar flow hood. The multi-well plate 207 is maintained at 37° C. in a humidified atmosphere of 5% CO₂ while the cells are plated. The procedure for plating cells is described subsequently in this application. Spare wells in the plate in which cells were not plated previously are filed with 100 μL of sterile distilled water to maintain 95-100% humidity inside the enclosed Chamber. The CO₂ Sensor is mounted on the right face of the Chamber Body 50 by tightening two 1½× 3/16-inch, hexnut-headed screws. The CO₂ line is attached using a quick connect fitting 72 to the ⅛ diameter nylon supply line. Next, the plate 207 is placed carefully into the inset on the bottom of the Chamber Body 50 and secured with a spring clip. The Chamber is enclosed by placing the Chamber Cover Gasket 56 in a groove on the top face of the Chamber Body and securing the Chamber Cover 52 in place on top of the Chamber Body and Chamber Cover Gasket by tightening sixteen 0.50×0.19-inch, hexnut-headed screws. Chamber assembly is completed by securing the two Heating Cartridges 62 into channels in side walls. of the Chamber Body from ports in the front face of the Chamber Body using one Heating Cartridge Retaining Screw 64 each.

Environmental control within the Biochamber 10 is maintained by regulating temperature and the partial pressure of CO₂ with two control systems. The RTD 58 is connected by insulated electrical wire to the input junction of the Temperature Controller 12. The two Heating Cartridges 62 are connected by insulated electrical wire to the output junctions of the Temperature Controller. The RTD 59 is connected by insulated electrical wire to the input junction of the Temperature Controller 17. The two Heating Cartridges 65 are connected by insulated electrical wire to the output junctions of the Temperature Controller, controlling the temperature of the table 18. The RTD 60 is connected by insulated electrical wire to the input junction of the Temperature Controller 15. The two Heating Cartridges 67 are connected by insulated electrical wire to the output junctions of the Temperature Controller, controlling the temperature of the humidity generating chamber 70. The CO₂ Sensor 66 is connected electrically to the input junction of the CO₂ Controller 14. The output gas stream from the CO₂ Sensor is connected to the CO₂ Supply Fitting 68 on the front face of the Chamber and the CO₂ Supply Tank with Regulator 16 connected to the input gas stream to the CO₂ Sensor. The assembled Biochamber 10 with environmental controls is allowed to thermally and atmospherically equilibrate for one to two hours before placement on the Motorized Stage 18. Temperature and pCO₂ are controllable to 37±0.5° C. and 5±0.2%, respectively, over the course of several days.

The Biochamber 10 with environmental controls next is secured on the Motorized Stage 18 with a spring mount. Cells for observation are chosen automatically by the software based upon user inputted parameters. For each well, one or more fields are selected. After selection of fields from up to preferably 96, 384 or 1536 (or more) wells for observation, the user initiates the automated imaging and analysis by selecting the appropriate option. Each field selected then is scanned sequentially at a user-defined interval, preferably between one and 60 minutes. It also is possible to scan at shorter or longer intervals depending on the requirements of a particular biological system. Each field is imaged under phase-contrast optics with transmitted light illumination using the Video-Rate CCD Camera 32 and under fluorescence optics with epillumination, using the Cooled CCD Camera 34.

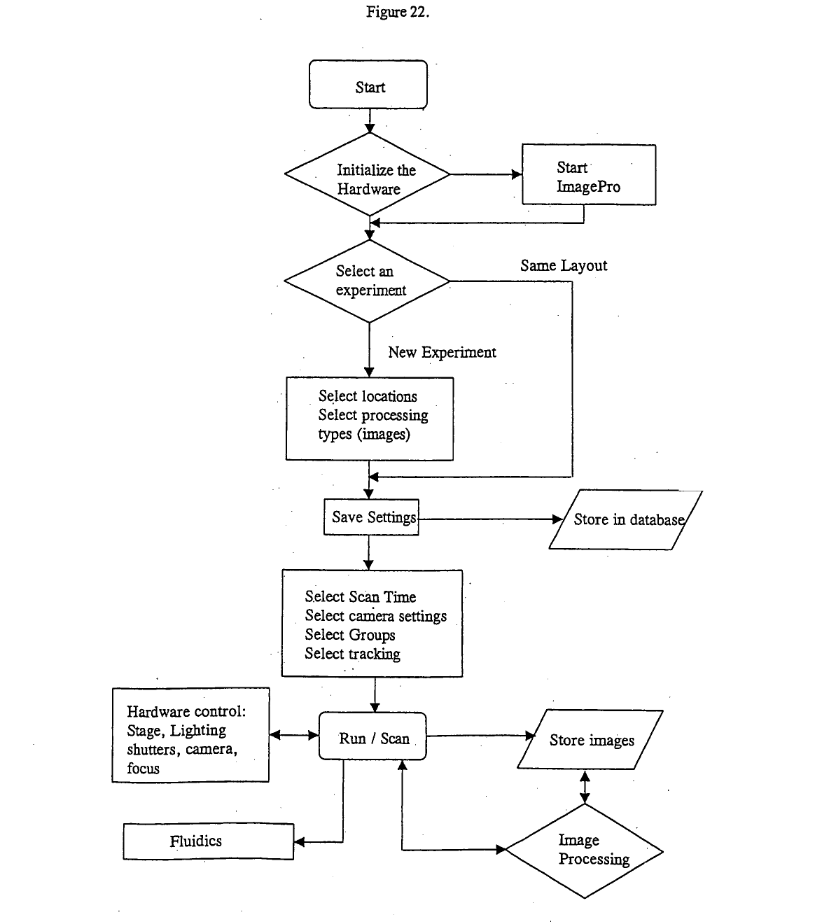

In a preferred embodiment, the dynamic state of each cell is evaluated using a robotic imaging system. Cells are observed using an Inverted Microscope 20 with extra-long working distance (ELWD) condenser and phase-contrast objectives and epifluorescence attachments. Digitized visible and fluorescence images of cells are obtained using a Cooled CCD Camera 34 connected directly to an interface board in the a Pentium 1.8 GHz PC. Imaging operations on the PC are performed using two software programs: ImagePro Plus, Version 3 (Media Cybernetics, Silver Spring, Md.) and CellMonitor, which has the functionality described in FIG. 22.

ImagePro controls the filter wheels, shutters and stage position through the serial interfaces of each module. CellMonitor communicates with ImagePro to run an experiment on the instrumentation. The program provides a user interface for viewing various locations on a plate. The operator determines-the position and focus. After all the locations are determined, the program sends commands to Imagepro to define a location (X,Y coordinates) and a focus position. Commands are then sent to the Lud1 controllers to locate a specific location and focus by sending specific instructions through the serial interface to the Lud1 controller. For a specific location, the operator can specify a visible image at a specific exposure. CellMonitor sends an instruction set to ImagePro to open the visible lamp shutter and to the camera to take an image. The image is displayed in both ImagePro as well as CellMonitor. The image is also saved to memory for later use. The location and name of the image is defined by CellMonitor, which instructs ImagePro to store the image. At each location, the operator may also require a fluorescent image. In this case, instructions are again sent to ImagePro to move the filter wheel to a specific location and close the visible light shutter and open the fluorescent shutter. The camera is instructed by CellMonitor through ImagePro to take an image and again display and store the image. The communication to the Lud1 controller is a serial set of instructions sent from ImagePro as instructed by CellMonitor. It is also possible to communicate directly to the Lud1 controller directly or by a pass through of commands to the Lud1 controller. This method is used to send multiple signals to the controller and overlap the stage movement with the filter wheel and shutter operations to speed up the operation. CellMonitor provides the sequence of events necessary to move to a location and take various images that are stored on the computer for later analysis of the images. The events are timed based of a required scan time or group of locations as well as by cellular events.

CellMonitor also provides image processing of an image if required by the operator. In one application of the system, a cell in a specific well can be tracked. Since cells move within the wells, it is possible for a cell of interest to move out of the view field of the image, if it is not tracked. The operator locates a cell of interest and the program takes a digital image. This image is a series of pixels each with a value from 0 (black) to 256 (white). This gray scale image represents the cell and surrounding background. A typical image is 658 (x coordinate) by 517 (y coordinate) pixels of information. Based on the magnification on the microscope, a pixel will represent a specific size in the plate. For example, at 20× magnification on the microscope, a pixel will represent 1 micrometer (micron) by 1 micrometer (micron). While cells vary in size depending on the type, a typical cell is about 10 microns in diameter. Using lighting methods common in microscopy, the edge of the cell, as well as the cell itself, can be adjusted to be brighter or darker than the background of the image. This is defined as contrast in the image. CellMonitor loads the recorded image. and translates it into an array of pixel values for a given location. By implementing various image processing techniques, the edge of the cell can be enhanced as the background is flattened or smoothed. The cell is then identified by locating objects in the image of a specified size or characteristic and rejecting all others. For example, a cell (object) should be 5 to 20 microns in diameter and be should be close to round. All other objects, irregular or too large are rejected. A second black and white image is then generated identifying likely objects in that image. Based on the location of the object (cell) in the previous image, the object in the current image is selected. The location is based on 2 parameters, location and cell area.

If the cell is moving, it will not be in the middle of the image. Therefore, the coordinates of the cell in the current image are used as the center location sent from CellMonitor to ImagePro for the next location. If the cell does not move out of the image by the time the next picture is taken, then the tracking/scan time is correct. This image processing of the image also can be used to detect a change in the cell characteristics. The cell can change shape, for example, before dividing. In that case the cell rounds up and then it divides into 2 objects. At that point, CellMonitor declares division. The center of the, well location is sent to ImagePro, to center the needle over the well where the cell has divided.

CellMonitor sends serial instructions to the needle drive to move to a specified location in the well to stain the cell. Cell staining involves removing liquid from a well and replacing that liquid with a second liquid containing an antibody. After incubation, the antibody is removed and a fluid used to dilute the stain. CellMonitor instructs the fluidics valves and syringe for these operations through serial instructions to the various modules. At various points in the process, positions are verified by optical sensors sent to the DataForth modules, to verify positions as well as to turn on and off pumps for cleaning and waste removal. These instructions are also serial instructions to the modules. After the staining/fluidics process, CellMonitor instructs ImagePro to take visible and fluorescent images of the cells, indicating the condition of the cell/cells. Both phase-contrast/no phase visible and fluorescence images are captured and processed then stored on the computer's hard drives.

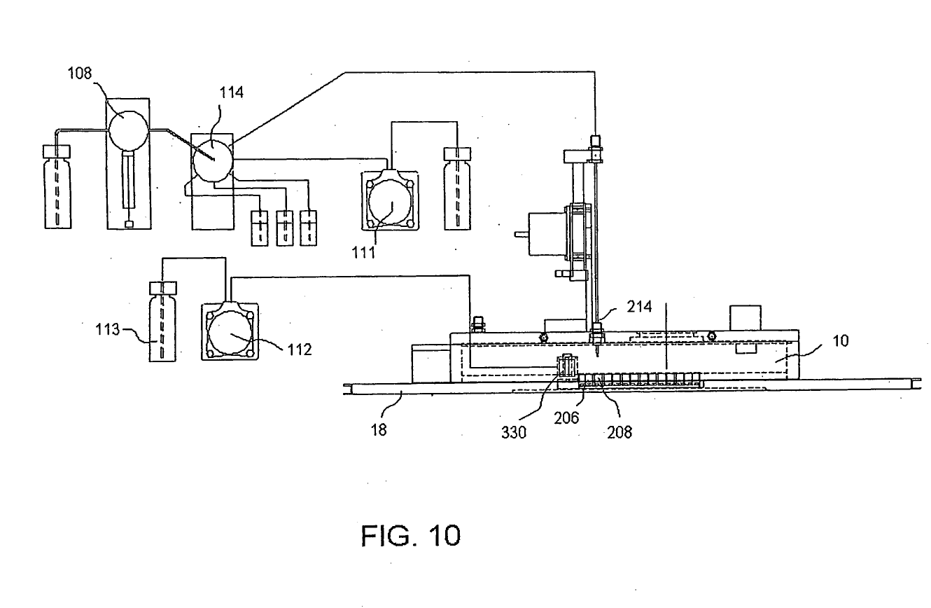

The robotic components of the imaging system (FIG. 10) are controlled by a Microscope Controller 28 which itself is controlled by commands from the PC, through an RS-232 interface. The Biochamber 10 is secured on a Motorized Stage 18 mounted on the Inverted Microscope 20. The Motorized Stage 18 has a resolution of 0.1 μm, an accuracy of ±6 μm, and a repeatability of 1 μm. Preferably, the Biochamber 10 itself with Motorized Stage 18 mounts directly on the Inverted Microscope 20. Focus control is achieved for each well using a Motorized Focus Drive Assembly and Controller 22 mounted on the focusing knob of the Inverted Microscope 20. Illumination is switched between transmitted light for phase-contrast imaging and epillumination for fluorescence imaging using a High-Speed Shutter for Transmitted Light 24 and a High-Speed Dual Filter Wheel with Shutter for Fluorescence 26. The Motorized Focus Drive Assembly and Controller 22, the motorized stage 18, the High-Speed Shutter for Transmitted Light 24, and the High-Speed Dual Filter Wheel with Shutter for Fluorescence 26 are connected electrically to the Microscope Controller 28. Initial x-y positioning of the Motorized Stage 18 stage and z-focal planes for each well are chosen by software and user programming on computer 42 or can be chosen using a Joystick 30 connected to the Microscope Controller 28.

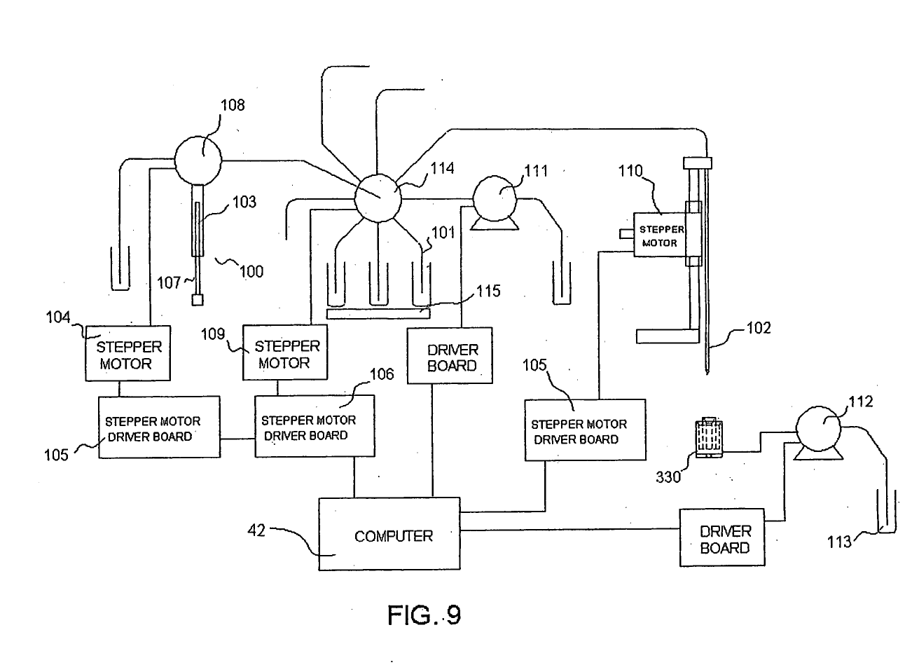

The z-robot pipette dynamically controls the composition of medium which bathes cells by automatically adding growth and/or quiescence factors to individual wells based on cell behavior. Software driving the operation of this z-robot pipette is integrated with software for monitoring cell behavior. (Refer to FIGS. 7-10) The system 300 also can add, remove or change medium based on external criteria, such as at particular time intervals chosen by the user. The z-robot pipette also transfers media from individual wells to supplemental analysis systems. The z-robot pipette for media exchange itself consists of a modified micropipette tip, see FIG. 9, mounted on a support arm driven by a z-axis stepper motor to move up and down and raise and lower the pipette tip for aspirating and dispensing media in 0.2 to 95% increments. Although 100 μL of medium typically is added to each 300 μL-volume well, aspirating all of the medium from a well can result in large shears being applied to the cells, which can detach or otherwise disturb them. Preferably, a minimum volume of 5 μL (corresponding to a depth of 125 m) of medium remains in each well at all times.

The major component of the pipetting system consists of a syringe pump 100 that can deliver growth factors, quiescence factors, or any type of liquid from multiple fluid reservoirs 101 through tubing to a pipette tip 102. The syringe pump consists preferably of a 250 microliter syringe 103 (although other syringe sizes can be used) that is driven by a stepper motor 104, which is in turn controlled via a multi-port stepper motor driver card 105 and a computer 42. The stepper motor 104 drives the plunger 107 of the syringe 103 up and down which results in a dispensing action (if the plunger is being driven into the syringe) or an aspiration action (if the plunger is being driven out of the syringe). The syringe is connected to one port of a distribution valve 108. The distribution valve can be from 3ports to 8 or more ports. One port is connected to the syringe 103, one port is connected to the pipette probe 102, one port to an optional wash pump 111, and the remaining ports to various fluid reservoirs 101. The distribution valve 108 is also stepper-motor driven through stepper motor 109 which can be driven also from stepper motor drive board 106. The syringe, stepper motor, stepper motor driver, and distribution valve can be obtained from Advanced Liquid Handling model MBP 2000 (Williams Bay, Wis.). A second distribution valve also can be mounted in the system in parallel with valve 108 to tie into more fluid reservoirs. The reservoirs 101 are thermostat to 4±2 C. by a thermostatting means 112, to allow good preservation of the growth and quiescence media and tied to the distribution valve 108 through 1/16 inch Teflon tubing.

The distribution valve (and thus the syringe pump) is plumbed via 1/16 inch-Teflon or stainless steel tubing to the pipette probe 102. The pipette consists of a stainless steel probe with an ID of 1/32 inch (0.031 inch) that narrows down to a tip D of 0.013 inch This pipette tip is used for both dispensing growth and quiescence factors into the 96 well plate as well as aspirating media out of the plate. The pipette probe has conductive coating on the outside of the probe that provides a signal that can be read by the computer 106. This electrical signal provides feedback on how much fluid there is in a well, consequently, when aspiration should stop. The pipette probe is driven in the “Z” direction by a stepper motor 110 that is tied into the stepper motor drive 105. This stepper motor drives the pipette probe up and down to dispense into or aspirate out of a selected well. The probe with conductive sensing can be obtained from Diba Industries, Inc., (Danbury, Conn.). The pipette stepper motor can be obtained from Advanced Liquid Handling model MBD Crawler (Williams Bay, Wis.). The pipette probe mounts into the biocontainment box by piercing through a Teflon bulkhead. The Teflon bulkhead has a hole in it that is sized to interference fit the OD of the pipette probe. Thus a seal is made between the OD of the pipette and the E) of the hole in the Teflon. This fit allows the pipette to move up and down freely and yet provides a seal to keep the environment within the Biochamber stable. The pipette moves down into the well to a depth of 3±1 mm from the top of the well for dispensing; the pipette moves down to the liquid surface in the well for aspiration (as measured by the conductive sensing mechanism on the probe tip); and the probe moves up out of the well with a clearance of 10 to 13 mm to clear the well as the well plate moves around on the x-y stage.

In an alternative embodiment, multiple dispensing/aspiration tips are utilized in parallel to dispense or aspirate a 96, 384 or 1536 well plate, thereby achieving higher throughput. A wash is performed to remove growth factors, quiescence factors or used media from the plumbing lines. The preferred wash fluid is Phosphate Buffer Saline (PBS). One approach is to use one of the reservoirs 101 for wash fluid to clean the system. Another approach is to use a separate wash pump 111 with the system. The wash pump 111 is a peristaltic pump with higher volumuetric flow capabilities that can be turned on by the computer 42 and pump through higher flows of wash fluid. The wash fluid is dispensed from the pipette tip 102 to a flush station within the Biochamber 10, as shown by item 330 in FIG. 9.

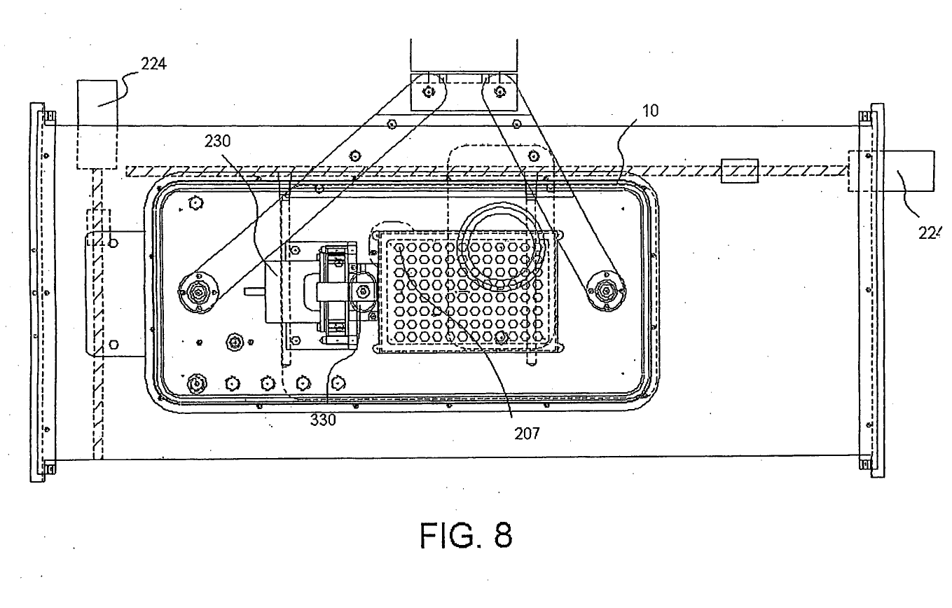

Fluid transfer in the Biochamber 10, involves location of the needle over a specified well in the plate. See FIG. 7. The needle is lowered into the well, and fluid is added or removed from the well. The needle then retracts and the table moves to another location under the needle or the needle is sent to the waste/cleaning station 330. See FIG. 8. The sterile fluid dispensed from the needle along with any waste fluids are sent to the waste vial 113, with a waste removal pump 112. Refer to FIG. 10 for a view of the fluidics components on the Biochamber.

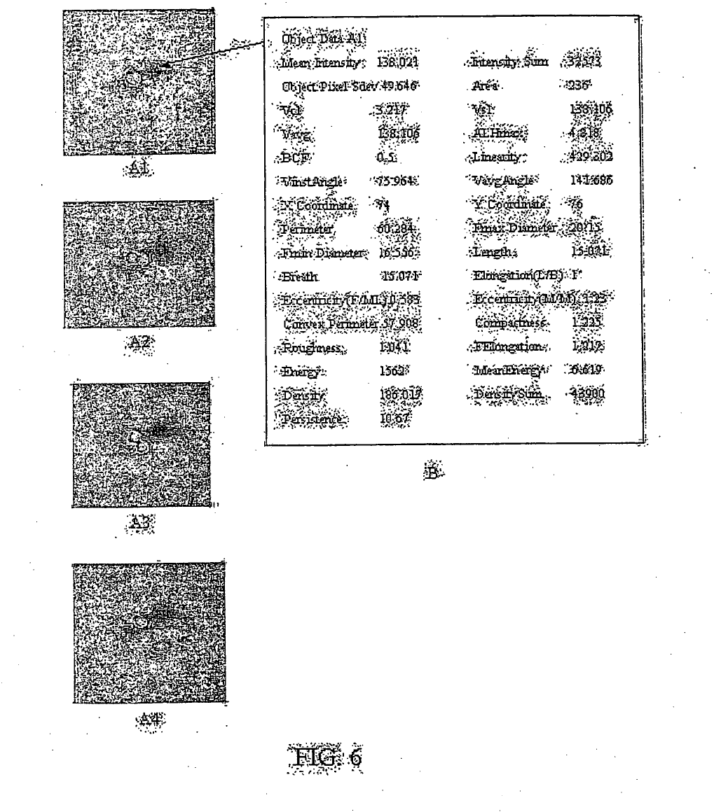

The occurrence of cell division and differentiation is detected by pattern recognition software. The software detects multiple other parameters including, but not limited to, 1) path of locomotion of a cell; 2) spread of cell movement 3) cell contact interactions in real time with other cells or objects; 4) and indirect cell responses (i.e., protein production). The number and two-dimensional shape (e.g., area and perimeter) of “objects” in each selected field are identified from phase-contrast images after application of an optical gradient transformation, thresholding, and dilation to detect each cell (see FIG. 6). Threshold values for shape parameters which indicate whether each object comprises one or more cells have been defined. The number of cells then is determined in each well at that particular time point by comparing the current values of the shape parameters with values for previous time points. Cell division is detected automatically as an increase in cell number between two time points. Image analysis also provides information on (x-y) positions which can be used to measure individual cell speed and directional persistence time by application of a persistent random walk model for migration, to determine the fraction of a population which is motile, and to adjust the position of the field to allow for cell movement while centering cells in the field.

The parameters of cell speed and directional persistence time for each cell, as well as the %-motile for a population of cells, are determined by fitting a mathematical model for a persistent random walk in an isotropic environment to observe data for the mean-squared displacement of each individual cell based on a time sequence of (xy1 position at the control of the cell). For example, see DiMilla et al. AIChE J. 38(7):1092-1104 (1992); DiMilla et al., Mater. Res. Soc. Proc., 252:205-212(1992); DiMilla et al., J. Cell Biol., 122(3):729-737. (1993); DiMilla, Receptor-Mediated Adhesive Interactions at the Cytoskeleton/Substratum Interface During Cell Migration, in CELL MECHANICS AND CELLULAR ENGINEERING (Hochmuth et al. eds., 1994); Thomas et al., Effects of Substratum Compliance on the Motility, Morphology, and Proliferation of Adherent Human Gliblastoma Cells, in 29 PROCEEDINGS OF THE 1995 BIOENGINEERING CONFERENCE, at 153 (R. M. Hochlmuth et al. eds., 1995), all of which are hereby incorporated by reference.

In determining the state of each cell over time, the imaging system can evaluate a variety of cell parameters concomitantly. In a preferred embodiment, measurement is made of over 65 parameters for each cell in each view field. Illustrative of such parameters are those detailed in Table I.

TABLE I
Parameters Measured

| | | Suggested | Type of | | |
|---|---|---|---|---|---|
| Measurement | | Name | Parameter | Description | Reference |
| 1. | Colony count | Object Count | Proliferation, | The number of objects in an image, where | (1-2) |
| | | | apoptosis | each object is a separated region within the | |
| | | | | image outlined on the basis of cell-like | |
| | | | | characteristics. | |
| 2. | Object count | Cell count 1 | Proliferation, | The number of individual cells in an image, | |
| | | | apoptosis | determined by dividing each object area | |
| | | | | (parameter 1) by a user defined average area | |
| | | | | for a cell. | |
| 3. | Proliferation | Cell count 2 | Proliferation, | The number of cells in a view field, | |
| | count | | apoptosis | determined by first determining the average of | |
| | | | | all objects within 3 times the preset preferred | |
| | | | | cell size. Then dividing each colony object by | |
| | | | | that average area to get a total cell count. | |
| 4. | Vinst(abs) | Instantaneous | Motility | The average of the Vinst values for all tracked | (1-2) |
| | | Speed | | cells in an image (see Vinst, below). | |
| 5. | Vinst(angle) | Instantaneous | Motility | The angle of the vector sum of the | (1-2) |
| | | Direction | | displacement of the cell position between the | |
| | | | | first and second points and between the | |
| | | | | second and third points. | |
| 6. | Vinst | Instantaneous | Motility | The vector sum of the displacement of the cell | (1-2) |
| | | velocity | | position between the first and second points | |
| | | | | and between the second and third points | |
| | | | | divided by the elapsed time between the first | |
| | | | | and third points. | |
| 7. | Vavg_inst(abs) | Instantaneous | Motility | The instantaneous speed of the average | (1-2) |
| | | Smoothed | | smoothed track through a specified number of | |
| | | Speed | | images before and after the specific image. | |
| 8. | Vavg_inst(angle) | Instantaneous | Motility | The angle of the instantaneous speed, #7. | (1-2) |
| | | Smoothed | | | |
| | | angle | | | |
| 9. | Vavg_inst | Average | Motility | The average of a specified number of images | (1-2) |
| | | Instantaneous | | of the smoothed track at a specific time/ | |
| | | Velocity | | image. | |
| 10. | Vsl(abs) | Straight Line | Motility | The straight-line velocity of the average | (1-2) |
| | | Speed | | smoothed track. | |
| 11. | Vsl(angle) | Straight Line | Motility | The angle of the straight-line velocity, #10. | (1-2) |
| | | Angle | | | |
| 12. | Vsl | Straight Line | Motility | The straight-line velocity of the instantaneous | (1-2) |
| | | Velocity | | speeds of the track. | |
| 13. | Vcl | Curvilinear | Motility | The change in the average velocity over the | (1-2) |
| | | Speed | | full track up to a specific field. | |
| 14. | Vavg | Average | Motility | The change in the average velocity of the | (1-2) |
| | | Velocity | | smoothed track to a specific field. | |
| 15. | Linearity | Linearity | Motility | The straightness of a cells motion, Vsl/Vcl. | (1-2) |
| 16. | Straightness | Smoothed | Motility | The same as linearity, using the smoothed | (1-2) |
| | | Linearity | | track, Vsl/Vavg. | |
| 17. | ALHmean | Amplitude | Motility | The measure of the oscillating amplitude of an | (1-2) |
| | | | | objects motion. The average amplitude of the | |
| | | | | track oscillations around the smoothed track. | |
| 18. | ALHmax | Maximum | Motility | The maximum amplitude of the oscillating | (1-2) |
| | | Amplitude | | component of the cells motion around a | |
| | | | | smoothed track. | |
| 19. | BCF | Beat Cross | Motility | The average number of oscillations about the | (1-2) |
| | | Frequency | | average track. | |
| 20. | Circular | | Morphology | A measure of the circular component of the | (1-2) |
| | radius | | | objects motion. | |
| 21. | Filtered | | Proliferation, | The number of objects that are filtered from | |
| | objects | | apoptosis | the analysis, based on their individual speed. | |
| 22. | % motile | Percent | Motility | The percentage of objects that is more motile | [1-2] |
| | | Motile | | than a given area per image. | |
| 23. | Elongation | Elongation | Morphology | The ratio of the length to the width of an | (3) |
| | (avg) | Rectangle, | | object based upon the ratio of the perimeter to | |
| | | Elongation | | the area in a rectangular model (Elongation | |
| | | Ellipse, | | Rectangle) or an elliptical model (Elongation | |
| | | Elongation | | Ellipse) or upon actual cell widths determined | |
| | | Feret | | throughout a set of angles (Elongation Feret) | |
| 24. | Start image | Track | Experimental | The first image for which a cell position is | |
| | | Segment Start | | included in a specific tracked. | |
| 25. | End image | Track | Experimental | The final image from which a cell position | |
| | | Segment End | | was included in a specific track. | |
| 26. | Cyte | Cyte | Morphology | An imaging position and an associated | |
| | | | | computer folder name used for acquiring and | |
| | | | | storing images. | |
| 27. | Avg Area | Average Area | Morphology | The average area of all the objects determined | (3) |
| | | Pixels or | | from an image. | |
| | | Average Area | | | |
| | | Microns | | | |
| 28. | Min Area | Minimum | Morphology | The minimum area (in pixels or microns) of | (3) |
| | | Area Pixels | | an object in a track or time series. | |
| | | or Minimum | | | |
| | | Area Microns | | | |
| 29. | Max Area | | Morphology | The maximum number of pixels or microns of | (3) |
| | | | | an object in a track or time series. | |
| 30. | Mean | | Morphology | The average gray scale intensity of the pixels | (3) |
| | intensity | | | within an object. | |
| 31. | Intensity Sum | | Morphology | The sum of all the pixel intensities within an | (3) |
| | | | | object | |
| 32. | Object Pixel | | Morphology | The standard deviation of the intensity of all | (3) |
| | SD | | | the pixels within an object. | |
| 33. | Area | Area Pixels | Morphology | The number of pixels in an object or the area | (3) |
| | | or Area | | in square microns of an object. | |
| | | Square | | | |
| | | Microns | | | |
| 34. | X coord | | Motility | The x coordinate of the center of an object in | (3) |
| | | | | an image. | |
| 35. | Y coord | | Motility | The y coordinate of the center of an object in | (3) |
| | | | | an image. | |
| 36. | Perimeter | Perimeter | Morphology | The sum of the pixels around the perimeter of | (3) |
| | | Pixels | | an object. | |
| 37. | Fmax | | Morphology | The maximum width of an object after the | (3) |
| | Diameter | | | angle is swept by a specified preset angle. | |
| 38. | Fmin | | Morphology | The minimum width of an object after the | (3) |
| | Diameter | | | angle is swept by a specified preset angle. | |
| 39. | Length | Length | Morphology | The maximum width of an object based upon | (3) |
| | | Rectangle | | fitting the perimeter and area to a rectangular | |
| | | | | model. | |
| 40. | Breath | Breadth | Morphology | The minimum width of an object based upon | (3) |
| | | Rectangle | | fitting the perimeter and area to a rectangular | |
| | | | | model. | |
| 41. | Elongation(L/ | Elongation | Morphology | The length/breath based upon fitting the | (3) |
| | B) | Rectangle | | perimeter and area to a rectangular model. | |
| 42. | Convex | | Morphology | The approximation of a convex hull of an | (3) |
| | Perimeter | | | object based on a swept angle. | |
| 43. | Compactness | | Morphology | The roundness of an object, perimeter squared/ | (3) |
| | | | (4 pi Area). | | |
| 44. | Roughness | | Morphology | Measure of the irregularity of the perimeter. | (3) |
| | | | | Perimeter/convex perimeter. | |
| 45. | FElongation | Elongation | Morphology | The Fmax/Fmin. | (3) |
| | | Feret | | | |
| 46. | Energy | | Morphology | A measure of the variation of the intensity of | (3) |
| | | | an object. | | |
| 47. | Mean Energy | | Morphology | The average variation in intensity of an object. | (3) |
| 48. | Density | | Morphology | The accumulation of the number of variations | (3) |
| | | | | divided by the area. | |
| 49. | Density Sum | | Morphology | The sum of all the variations within an object. | (3) |
| 50. | Unique Track | | Cell-specific | A unique number for each track generated | |
| | Index | | Delimiter | from cell-like objects in a series of images. | |
| 51. | Track Size | | Cell-specific | The length of a track in terms of the number | |
| | | | Delimiter- | of cell positions included. | |
| | | | Motility | | |
| 52. | Track | | Cell-specific | The larger of the x or y displacements, in | |
| | Boundary | | Delimiter- | pixel widths, of cell positions along a track. | |
| | (pixels) | | Motility | | |
| 53. | Fluorescent | | Selected | The intensity sum of an object, based on a | (4) |
| | marker 1 | | protein | fluorescent marker, TRITC. | |
| | | | expression | Note: Filter sets for detecting various | |
| | | | marker for | fluorophore can be purchased from: Chroma | |
| | | | phenotype | Technical Corp. 72 Cotton Mill Hill, Unit A9 | |
| | | | | Brattleboro VT 05301, USA | |
| 54. | Fluorescent | | Selected | The intensity sum of an object, based on a | (4) |
| | marker 2 | | protein | fluorescent marker FITC. | |
| | | | expression | | |
| | | | marker for | | |
| | | | phenotype | | |
| 55. | Fluorescent | | Selected | The intensity sum of an object, based on a | (4) |
| | marker 3 | | protein | fluorescent marker DAPI. | |
| | | | expression | | |
| | | | marker for | | |
| | | | phenotype. | | |
| 56. | Fluorescent | | Selected | The intensity sum of an object, based on a | (4) |
| | marker 4 | | protein | fluorescent marker CY5. | |
| | | | expression | | |
| | | | marker for | | |
| | | | phenotype | | |
| 57. | Proximity | | Cell-cell | The number of cells of Type A that interact or | |
| | (Cell to cell | | interactions | touch a second cell of Type B, based on a | |
| | contact) | | (e.g., antigen | distance from the perimeter parameter of cell | |
| | | | presentation) | Type B. | |
| 58. | Frequency of | | Cell-cell | The rate of cells of type A coming into | |
| | Proximity | | interactions | proximity with a cell of type B. | |
| | | | (e.g., antigen | | |
| | | | presentation) | | |
| 59. | Duration of | | Cell-cell | How long the cells of type A stay in contact | |
| | Proximity | | interactions | with a cell of type B. | |
| | | | (e.g., antigen | | |
| | | | presentation) | | |
| 60. | Cell-Specific | | Cell-cell | The number of cells interacting with a second | |
| | Proximity | | interactions | cell of a specified morphology. | |
| | | | (e.g., antigen | | |
| | | | presentation) | | |
| 61. | Phagocytosis | | Cell-cell | The number of fluorescent beads (antigens) | (5) |
| | Attachment | | interactions | that are attached to a cell. | |
| | | | (e.g., antigen | | |
| | | | presentation) | | |
| 62. | Phagocytosis | | Cell-cell | The number of fluorescent beads (antigens) | (5) |
| | Engulfed | | interactions | inside a cell. | |
| | | | (e.g., antigen | | |
| | | | presentation) | | |
| 63. | Phagocytosis | | Cell-cell | The area of fluorescent beads (antigens) that | (5) |
| | Attachment Area | | interactions | are attached to a cell. | |
| | | | (e.g., antigen | | |
| | | | presentation) | | |
| 64. | Phagocytosis | | Cell-cell | The area of fluorescent beads (antigens) inside | (5) |
| | Engulfed | | interactions | a cell. | |
| | Area | | (e.g., antigen | | |
| | | | presentation) | | |
| 65. | Persistence | | Motility | Based on the random walk model, the time a | |
| | | | | cell proceeds in a given direction at a | |
| | | | | consistent speed. | |

Note that for parameters 53 through 56—Fluorescent markers—explanations are given in Table 1 of prominent fluorescent markers. This invention can use any type of fluorescent markers that can be added based upon the availability or design of the specific markers and the availability or design of specific filters that allow that fluorescent output to be detected. Although four florescent outputs are shown in Table 1, filter set combinations can be purchased or designed that allow eight or even twelve simultaneous florescent markers to be used and detected in this invention.

Data acquired from thousands of recorded images provide quantitative information regarding the kinetics of cell movement, cell division, apoptosis, morphology, adherence, and physiologic function. The kinetics of each assay can be measured typically to a resolution of “minutes” and “per unit cell.” Population studies yield information on cell-cell synergistic effects, the fraction of cells responsive and group thresholds. Motility assays provide cell movement over time, direction, and cell phenotype.

According to one embodiment of the invention, apoptotic and mitotic events are detected with visible light images. Apoptotic cells are refractile for a much longer period of time than mitotic cells. By detecting the “bright refractile” objects in the image and examining the track lengths, i.e., the amount of time the “bright refractile” object persisted from image to image, produced by these objects, the frequency of apoptotic events can be determined. Data analysis software produces track length data for every track (cell) and exports the information to a database. As cells undergoing apoptosis consistently possess longer track lengths than normal cells, the software can readily detect apoptotic events by identifying “bright refractile” objects with long track lengths.

The same technique can be used to automatically count mitosis. Cell division produces short track lengths. Since there is a certain amount of back ground noise generated when cells move, the track lengths used for mitotic events must be longer than the tracks of the background and shorter than the tracks of apoptotic events. This technique provides a more accurate account of cell proliferation than counting total cells in a view field, which often yields inaccurate estimates when large numbers of cells are migrating in or out of the view field.

A data set, representing the various parameter values recorded during the experiment, is generated for each cell. The data can be presented for evaluation in a variety of formats. Combinatorial and multi-parametric assays yield highly informative results. Two-dimensional plots reveal cell sub-population responses and offer useful perspectives, often revealing subtle or unexpected responses, which can be referred to as “unexpected biology.” A database of results, comprising the various data sets, is automatically constructed to allow further data mining as additional mathematical analyses are devised.

The combination of data sets from various disease-model cells is analyzed by bioinformatics software, which automatically compiles a knowledge base of protein, cellular and molecular relationships. The knowledge base enables scientists to ascertain protein function and to conduct in silica testing, using computer modeling. Upon completion of the data analysis, the system can generate a report summarizing the findings.

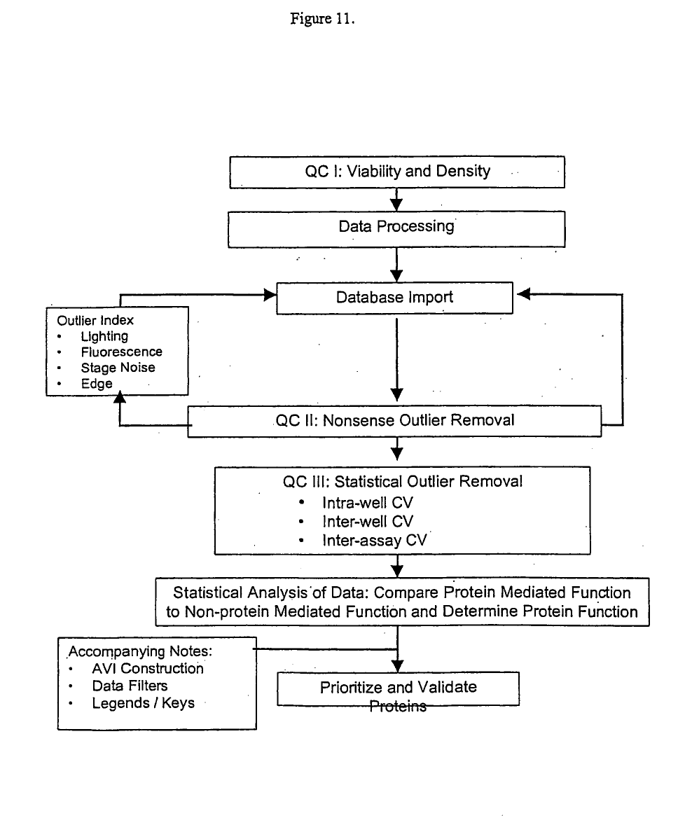

An exemplary data analysis scheme is depicted in FIG. 11. After the data acquisition, a Quality Control Step I (QC I) is performed. This step statistically evaluates the viability and density of the cells. Tests also verify that the sampling rate/resolution is sufficient for suitable motility measurement. The cells within a specific assay must be viable, i.e., growing and functioning normally, and must have a density (how close or far away cells are from one another) such that the image acquisition can provide appropriate information. If these criteria are not met, the assay is adjusted, for example, by increasing the sampling rate or by repeating the test with suitable cells before, image analysis and processing.

The data analysis system processes the sequential images, both visible and fluorescent, to identify the cells within the image and to quantify the multiple parameters for each cell within each image. The image processing software quantifies the parameters of Table I for each cell within the specific viewfield of the imaging system. Each cell is tracked from one frame to the next image and related to one another through its track. This tracking is accomplished by the software selecting a “given” cell in the first image and quantifying all the parameters of Table I for that cell. Then the software selects a set of cells in the second image that are in proximity to the given cell of the first image in terms of x-y positioning. The software determines all the parameters of Table I for the selected set of cells in the second image and compares those parameters of the given cell of the first image. The software then selects one cell from the second image as statistically the same as the given cell of the first image. This threshold of statistically similarity can be set at different levels of statistical confidence, such as 95, 99, or 99+ percent. If the software does not detect the chosen level of similarity, then that track is stopped at the fist image. The level of statistical similarity can be increased by acquiring images at more closely spaced times. All of the parameters of Table I are calculated for each cell within the image viewfield. In the database-import step, the processed data are exported from the processing software and imported into a database. The database stores all of the separate mathematical parameters from each cell, in each well or in each view frame.

Next, in the Quality Control Step II (QC II); the system identifies and removes “nonsense” outliers from the data sets. A number of factors may produce nonsense outliers, such as mechanical irregularities of the visible or fluorescent lighting, mechanical stage noise from the XY stage, sample well edge distortion, and power outages. After the software identifies the outliers, a technician reviews the excised outliers and removes the data from the database. Alternatively, the data can be re-processed and then re-imported into the database.

In the Quality Control Step m (QC III) statistical outliers are removed from the data. Statistical outliers represent real data but, for statistical precision, are removed from the data analysis. Statistical outliers are identified using established methods such as Z-scores or MAD scores. See e.g. Robert R. Sokal & F. James Rohlf, BIOMETRY, THE PRINCIPALS AND PRACTICE OF STATSTICS IN BIOLOGICAL RESEARCH, 3^{rd} Edition, W.H. Freeman and Company, New York.

Next, the system conducts a statistical analysis of the data. Protein-, chemical- or biological-mediated wells are compared to control wells, and significant parameter changes are identified and analyzed. As the system identifies significant changes in variety of parameters in Table I, it provides a wealth of information regarding the physiological effects and, hence, the function of proteins of interest. Thus, by comparing the kinetic data from the exposed cells with various controls, the system elucidates the function of a protein in one or more disease models. This information is then used to prioritize the proteins in a library. The proteins are prioritized by ranking the statistical difference in parameters between the protein. mediated well and the control biology well. The parameters used to prioritize the proteins depend upon the disease model and the parameters that are most indicative of the disease state. Alternatively, methods such as cluster analysis can be used to stack rack a number of the protein parameters concurrently. The data produced also validates the function of the specific protein of interest in terms of the disease model and the protein's relationship to a disease or healthy state in humans.

The inventive methodology also can provide useful information regarding the disease model itself. In this regard, identification of the significance of a previously overlooked or unappreciated parameter, so called “unexpected biology,” can greatly enhance the understanding of a disease model and provide a foundation for additional research.

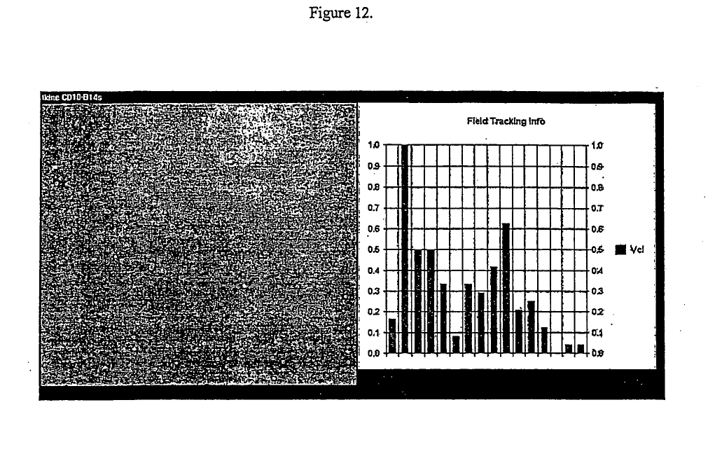

In addition, the system enables the identification and characterization of subpopulations. For example, FIG. 12 illustrates an evaluation of subpopulations of T lymphocytes. In this figure, an assay was conducted using T lymphocytes as a disease-model cell. All of the parameters in Table I were measured for up to 72 hours. The left panel of FIG. 12 shows a single time image of the T lymphocytes. The histogram pictured in the right panel shows two distinct subpopulations of the T lymphocytes. The Y-axis is the population frequency and the X-axis is a fraction of the cells segregated by curvelinear velocity. Thus, the cells are segmented into slow movers (to the left of the histogram) and the fast movers (to the right of the histogram). Any of the parameters of Table I can be screened for subpopulations. Thus, multiparametric analysis extends the breadth of information obtainable and increases assay sensitivity.

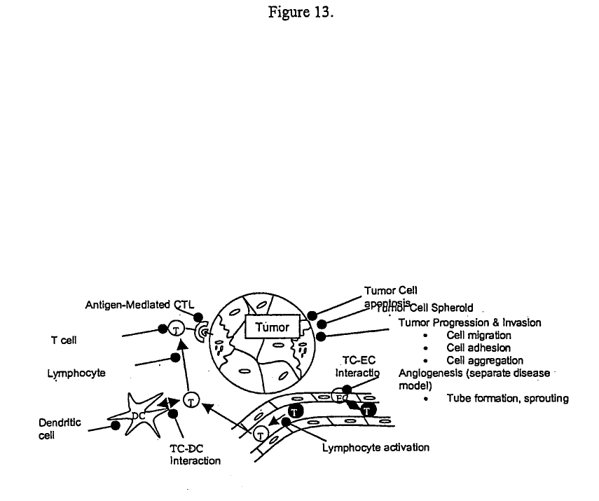

A variety of disease-model cells can be used in the assays of the present invention to elucidate protein function. For example, the oncogenesis disease model can be used to elucidate a protein's function with respect to specific components of oncogenesis. FIG. 13 provides schematic of the disease model. Besides a cancer cell-line of interest, the model encompasses T-lymphocytes, B-lymphocytes, natural killer cells, dendritic cells, monocytes and macrophages. Assayed components include antigen-specific tumor cell killing, tumor cell apoptosis, and various components of anti-tumor immunity, such as antigen presentation by dendritic cells and T lymphocyte recruitment. Quantitative endpoints include the stimulation or suppression of cell migration (chemotaxis), cell proliferation, and cell-cell interaction and the stimulation or inhibition of cell death. The discovery of a statistically significant effect establishes a functional role in oncogenesis for the protein of interest. All of the parameters in Table I are measured for each cell and type of Gell in the disease model.

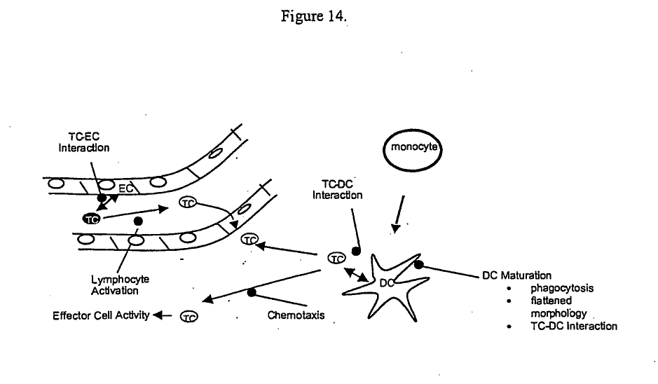

In another example, the primary immune response disease model detects the function of a protein with respect to specific components of immune disease. See FIG. 14. Exemplary immune diseases include, but are not limited to, inflammatory diseases, such as rheumatoid arthritis and inflammatory bowel diseases, and autoimmune diseases, such as Type I diabetes, multiple sclerosis and lupus. Relevant cell lines include T-lymphocytes, B-lymphocytes, natural killer cells, dendritic cells, monocytes, macrophages, and cell-lines that are relevant to the immune disease under consideration. The relevant quantitative endpoints can be similar to those identified for oncogenesis, relating to cellular chemotaxis, cell proliferation, etc. The role of candidate proteins in the effector phase of immune cell function, e.g., tumor cell killing, also is assayed. The functional maturation and differentiation of various immune cells also can be assayed for hundreds of cells at a single-cell resolution level. The discovery of a statistically significant effect establishes a functional role for the protein of interest in the immune disease.

In addition, so-called “secondary immune response” models can be created. Examples include, but are not limited to, 1) comparing T cell and B cell responses after antigen challenge, 2) comparing the functionality of a patient's cells with a control population of similar cells (e.g., dendritic cells; T or B lymphocytes, etc.), 3) observing responses to “blocking factors” or drugs, and 4) evaluating the effect of stimulating or suppressing the patterns of immune response (i.e., the phenotypical outputs as measured by the invention) by the presence of known or unknown proteins or drag candidates.

Such assays serve to categorize responses in patterns that define certain disease states. The responses can be compared to pretreatment data to determine the success of a therapy or to pools of data from cell samples from a variety of individuals to define disease subtypes and response patterns. Such data is useful not only to researchers but also to clinical practitioners for patient diagnosis, treatment and follow up.

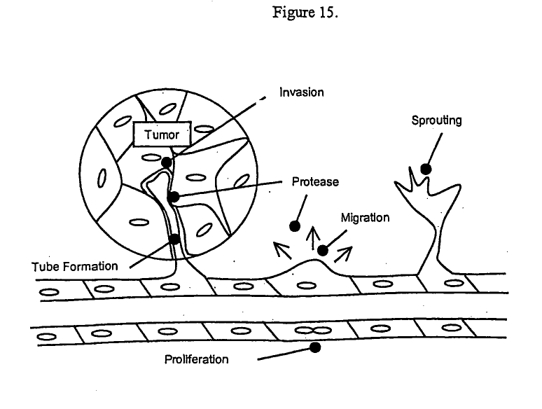

In yet another embodiment of the invention, the angiogenesis disease model elucidates protein function with respect to specific components of angiogenesis, which is the process of developing new blood vessels (see FIG. 15). Angiogenesis may be a desirable objective, as is the case with neovasculature of a transplanted organ, or it may be undesirable, as with the neovasculature of a tumor. Accordingly, the discovery of proteins that stimulate or repress angiogenesis can be instrumental to handling a variety of potential pathologies associated, for instance, with organ transplantation, atherosclerosis and oncogenesis, respectively.

Angiogenesis involves a series of steps undertaken by endothelial cells. In order to form a new blood vessel, endothelial cells of existing vessels must proliferate, sprout, invade the immediate vessel environment by protease-mediated migration, invade the new site and form the novel blood vessel. Each of these steps can be measured quantitatively using in vitro assays and combined into multiparametric assays. Quantitative endpoints include endothelial cell migration, proliferation and morphological changes, such as sprouting. In addition, bioassays such as the formation of fluid-filled tubes, protease-mediated extracellular matrix digestion and target organ invasion also can be performed. The discovery of a statistically significant effect establishes a functional role for the protein of interest in the angiogenesis-related disease.

Alternative embodiments utilize expanded disease models that can include additional assays conducted for an existing disease model. Disease states can be categorized and staged by similarity of response patterns. For example, patients can be defined as having a certain disease, disease in remission, or recurrent disease based on response patterns. Disease models can be combined to study, for example, common aspects of multiple disease states, such as inflammation. Moreover, the models can continue to be developed by tying genotype to phenotype to disease outcome and by including more disease areas. Also, traditional protein-protein interaction assays, such as phage display and two hybrid screening, can be employed generally in the claimed invention.

In a preferred embodiment, more than one disease-model cell is evaluated in a single experiment. The disease-model cells can be from the same disease model, or separate ones. Alternatively, a single run of the instant method can comprise multiple disease-model cells from multiple disease models. For example, a first disease-model cell of the inventive method may be from a primary immune response disease model and evaluate a protein's function in a co-culture environment. A second disease-model cell also may be from a primary immune response disease model, but may evaluate a protein's role in maturation. Alternatively, the second disease-model cell may be from a different disease model, such as the angiogenesis model. In yet another embodiment, the instant protein-analysis method can comprise, in a single run, multiple assays from multiple disease models or multiple assays that cover various parts of one disease model.

The instant invention is suited ideally for combinatorial experiments. A combinatorial experimental approach is where a large number of different experiments, each with different parameters, are performed in one experiment producing many results in parallel. Such a design can encompass a variety of disease models, assays, specific cell lines, protein targets, media and experimental parameters. Thus, a single run employing a plate with 96, 384, 1536 or more sample wells can evaluate a protein by means of a variety of disease models, wherein a multitude of assays are performed for each disease model and a myriad of parameters are measured for each assay.

In another embodiment, the present invention provides methods and compositions for identifying lead targets for development, i.e., proteins that have functions of interest. In this regard, a plurality of proteins can be examined simultaneously by the disclosed automated system. Ideally, the plurality is evaluated using a combinatorial design, such that each protein is evaluated using a variety of disease models, wherein a multitude of assays are performed for each disease model and a myriad of parameters are measured for each assay. In this fashion, the automated system identifies particular proteins within the plurality that have functional traits of interest. Alternatively, the plurality of proteins can be added to one sample well, such that the plurality of proteins is studied in a disease model and the effects. of the plurality of the proteins are identified. If a desired effect is identified, the plurality (or pool) of proteins can be deconvoluted by splitting the plurality into in a smaller number of pluralities and re-running those pluralities through the disease model, or by splitting the plurality into singular proteins and re-running those proteins through the disease model. In either case, the pool is deconvoluted to the point where the proteins of interest are identified.

Protein libraries can be created from a variety of sources, including cDNA, protein chips, culture supernatants, transgenes, novel peptides, disease-specific sera and cell lines, and antibody libraries. Purified proteins and antibodies can be added directly to the culture medium. Alternatively, a given protein can be studied in its relevant cellular context by introducing the encoding polynucleotide into the cell type in question or by introducing the protein directly into the cells, as described below.

In one embodiment, a protein of interest is brought into contact with a disease-model cell by inserting the protein's gene into the cell, for example, by retroviral transduction or lipid-mediated transfection. Depending on the assay, cDNAs and other constructs are introduced either stably or transiently. Typically, clones of novel or potential candidate target molecules are prepared in single plasmid arrays and introduced into cells. cDNA sequences encoding potential target proteins are identified by sequencing and inserted into retroviral transfection systems for development. of permanent cell lines that produce the target protein. Any technique that transduces or transfects genes or proteins into cells may be used in this context. See Sambrook et. al., 1989, MOLECULAR CLONING, A LABORATORY MANUAL, Cold Spring Harbor Press, NY; and Ausubel et al., 1998, CURRENT PROTOCOLS IN MOLECULAR BIOLOGY, Green Publishing Associates and Wiley Interscience, NY.

Other transfection methods can be employed as needed. For example, the lenteviral system can be used for nuclear delivery of a cDNA in resting cells or cells that have stopped dividing due to differentiation. Adenoviral vectors can be used when nuclear delivery is not crucial and cells are resting. In first-pass screening, or when assay endpoints are brief (less than 3 days), lipid-mediated transfection is sufficient. In other embodiments, genes can be “knocked out” by means such as antisense or inhibitory RNAs or dominant negative. mutation, or by the use of heterologous, inducible promoters.

The present invention is described further by reference to the following example, which is illustrative only.

### EXAMPLE

### Use of a Primary Immune-Response Disease Model

A. Primary Immune Response Assay 1

This assay determines whether a protein is involved with dendritic cell maturation and the interleukin-1 (IL-1) pathway of the primary immune response. Under natural conditions, dendritic cells mature and lose viability. Remaining cells consume the expired cells through phagocytosis. Thus, phagocytosis is indicative of dendritic cell maturation and differentiation.

Interleukin-1 beta was evaluated via this disease-cell model. Dendritic cells (DCs) were generated from peripheral blood monocytes by culture in IL-4 and GM-CSF. For 24 hours the DCs then were incubated with 2-micron fluorescent polystyrene beads in the presence (panel A) or absence (panel B) of IL-1 beta (20 ng/rnl) and tumor necrosis factor (TNF). Fluorescent images were superimposed upon visible light images to align clusters of phagocytized beads with phagocytic DCs.

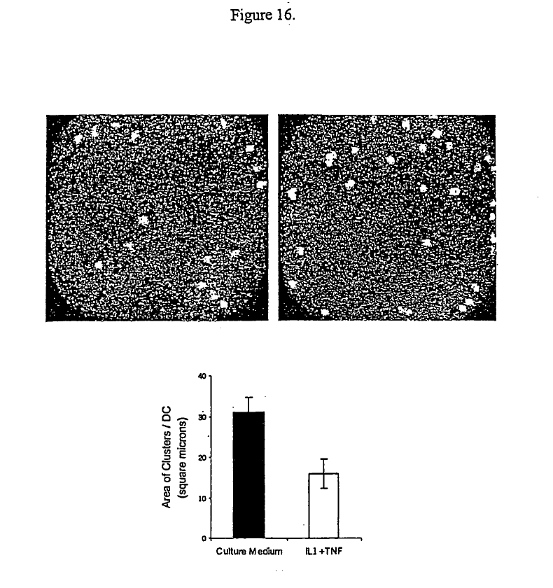

Panels A and B of FIG. 16 depict all fluorescent beads with larger clusters arising from the phagocytosis of beads by DC. Cells containing fluorescent bead clusters of area greater than 60 square microns from duplicate wells are quantified in panel C.

As shown in FIG. 16, IL-1 beta decreased the amount of phagocytosis in DCs. The effect of IL-1 can be separated from the effect of TNF by the use of the appropriate positive and negative controls, such as incubation with either IL1 or TNF alone. Thus, the PIR-1 assay is an effective tool for determining whether a protein is involved in the IL-1 pathway and dendritic cell maturation and differentiation.

B. Primary Immune Response Assay 2.

This assay, a second example of a primary immune-response disease model, evaluates a protein's function in a co-culture environment. In particular, the assay evaluates a protein's capacity to influence dendritic cell-T cell interactions. The interaction of T lymphocytes with antigen presenting cells, especially dendritic cells, is an important step in antigen presentation.

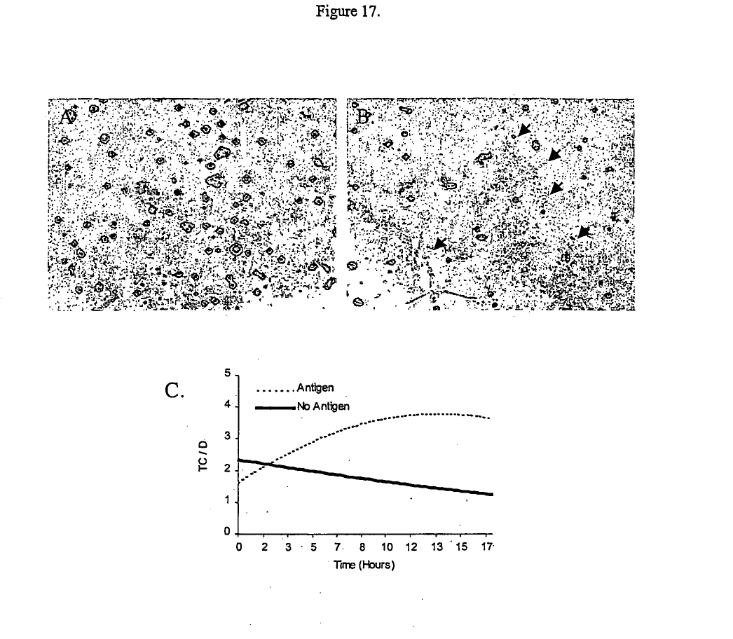

DCs were cultured with T lymphocytes and exposed to the protein of interest, Staphylococcal Enterotoxin B. DCs were generated from peripheral blood monocytes by culture in IL-4 and GM-CSF. The cells were co-cultured with naive T cells for 24 hours and imaged every 3 minutes in the presence (FIG. 17, panel B) or absence (panel A) of 1 ng/ml superantigen Staphylococcal Enterotoxin B. Lymphocytes were distinguished from dendritic cells using CytoWare® image analysis software. In FIG. 17, the number of T cells (TC) within a single T cell diameter (see arrows, no outlines) of a dendritic cell (DC) were quantified for each image and plotted per DC in panel C. T cells that were not located proximal to a dendritic cell are outlined.

FIG. 17 demonstrates that Staphylococcal Enterotoxin B influences dendritic cell-T cell interactions. These results confirm the assay's utility in identifying autoimmunogenic proteins, inflammatory agents and vaccine candidates.

C. Primary Immune Response Assay 3

This assay, a third example of a primary immune-response disease model, evaluates a protein's role in DC maturation. Changes in DC morphology, such as the ratio of cell length to breadth and spreading are indicative of DC maturation. Such changes are believed to arise from the secretion of cytokines, e.g., interferons, TNF, etc., resulting from antigen-specific TC-DC interactions.

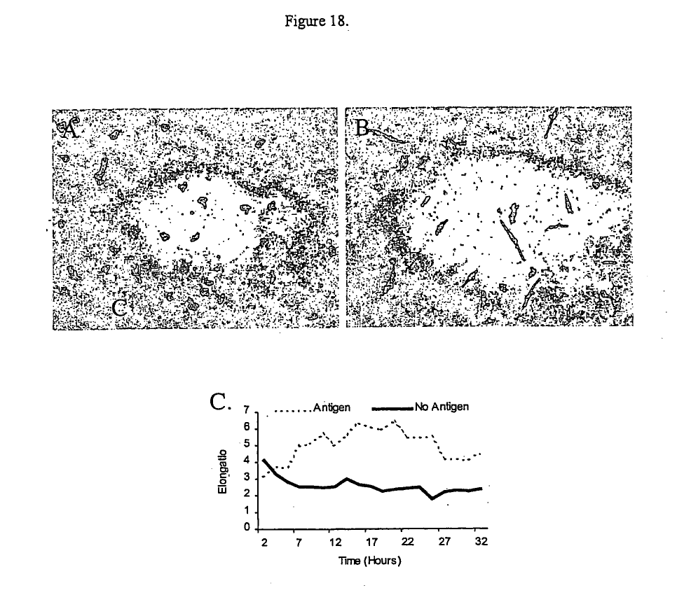

In this assay, DCs were cultured with T lymphocytes in the presence FIG. 18, panel B) or absence (panel A) of Staphylococcal Endotoxin B, the protein of interest. DCs were generated from peripheral blood monocytes by culture in IL-4 and GM-CSF. The cells were co-cultured for 24 hours with naive T cells (TC) and then were imaged every 3 minutes, with or without Staphylococcus Enterotoxin B superantigen (1 ng/ml). Lymphocytes were distinguished from dendritic cells using CytoWare® image analysis software.

The ratio of cell length to breadth was calculated for every cell in each image. The image averages, presented in panel C of FIG. 18, show that the superantigen induced dendritic cell elongation. Accordingly, the assay provides an effective and sensitive means for evaluating the function of a protein with respect to antigen presentation, lymphocyte activation, dendritic cell maturation, and involvement with signaling pathways.

D. Primary Immune Response Assay 4

This assay, a fourth example of a primary immune-response disease model, evaluates a protein's effect on T cell activation by analyzing parameters such as cell migration. As dose-dependent increases in lymphocyte migration are indicative of lymphocyte activation, the assay elucidates protein function in pathways connected with lymphocyte activation, such as the interleukin 2 (IL-2) pathway. Such pathways play important roles in inflammation and autoimmune diseases, such as rheumatoid arthritis and multiple sclerosis. The motility assay also is useful for establishing protein function in metastasis, angiogenesis, wound healing and tissue remodeling.

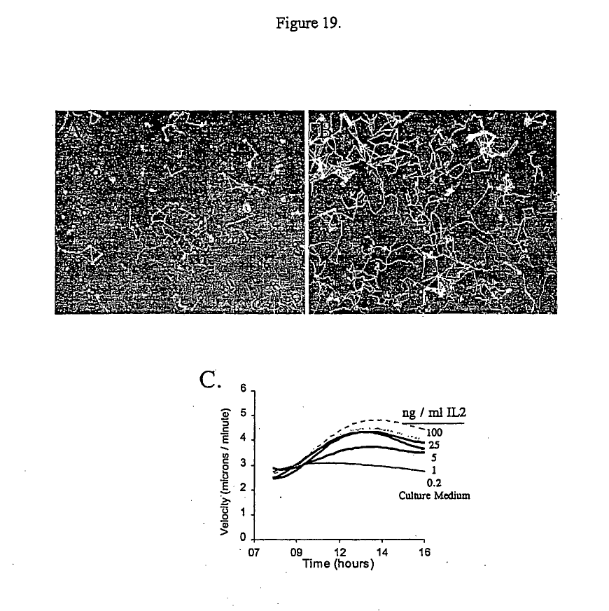

Primary lymphocytes were isolated from peripheral blood and cultured in the presence (FIG. 19, panel B) or absence (panel A) of IL-2, the protein of interest, at various concentrations (0.2, 1, 5, 25, and 100 ng/ml) for the indicated time periods. Lymphocyte migration was quantified from single cell tracking using CytoWare® image analysis software. These data confirm the role of IL-2 in lymphocyte activation. As shown in panel C, IL-2 produced a dose dependent increase in lymphocyte migration, confirming its role in lymphocyte activation.

E. Primary Immune Response Assay 5

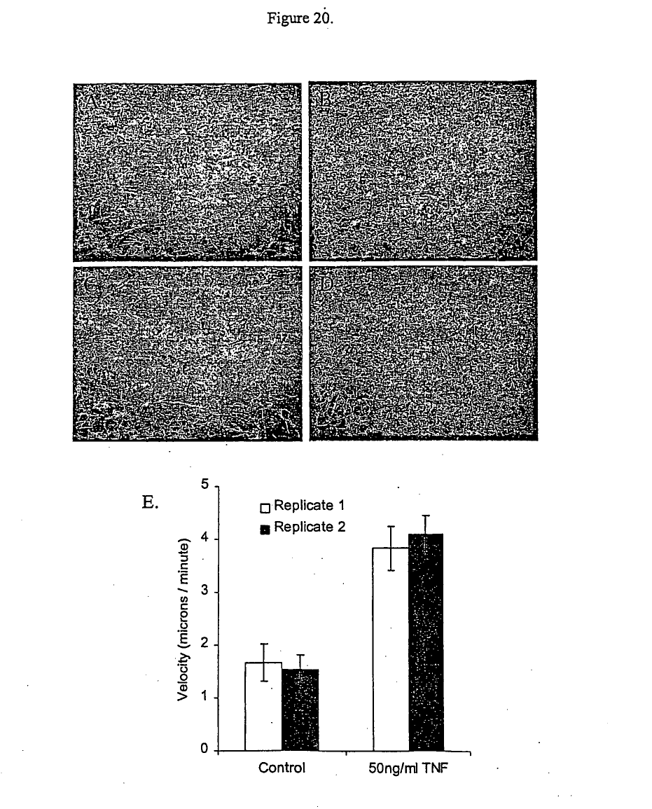

In a fifth example of the primary immune response assay, the effect TNF-alpha on dendritic cell migration was determined. DCs were generated from peripheral blood monocytes by culture in IL-4 and GM-CSF. In duplicate wells, cells were cultured in the presence (FIG. 20, panels B and D) or absence panels A and C) of 50 ng/1 ml of TNF-alpha. Cells were imaged every two minutes in each of the wells. The accumulated tracks for more than 300 images are shown in FIG. 20 with light lines. The average velocities for the cells over the period are plotted panel E), with error bars representing standard deviation.

As FIG. 20 shows, TNF-alpha induced cell migration of DCs. Since DC motility is indicative of cell maturation and differentiation, the assay demonstrates TNF-alpha's role in the DC maturation. Because mature DCs play a central role in antigen presentation during a primary immune response, the assay assists practitioners in identifying proteins active in immunopathologies.

F. Concurrent Assays

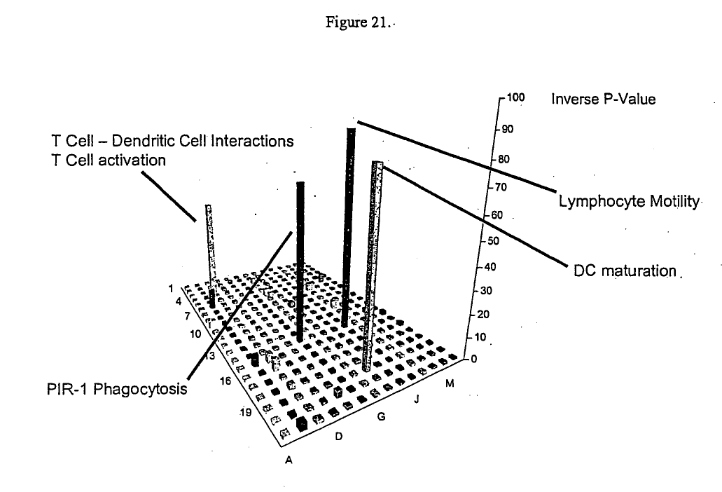

The above assays can be performed concurrently in one cell culture plate, as shown in FIG. 21. In many cases, more than one assay can be performed within the same well. For example, FIG. 21 demonstrates T Cell—Dendritic Cell Interaction and T Cell activation occurring in a single well. The ability to combine a variety of disease model assays into one cell culture plate improves throughput, productivity and sensitivity. For example, by measuring both lymphocyte speed and direction of travel in the presence of DC, it is possible to show lymphocyte migration to specific DC for antigen presentation and subsequent TC proliferation at that DC—all within a single well. The assays and outputs of the previous examples A through D above can all be performed in the single plate of FIG. 21.

TABLE II
Components of Automated Single-Cell Culture System depicted in FIG. 1.

| Component # | Name | Manufacturer | Description |
|---|---|---|---|
| 10 | Chamber | Machine Shop | Parts described in Table III as components #50-92. |
| 12 | Temperature | Omega | Model CN76000. Input from RTD (#58 in Table |
| | Controller | | III); output from two heating cartridges (#62 in |
| | | | Table III) |
| 14 | CO₂ Controller | Omega | Model CN 76000. Electrical input from sensor |
| | | | (#66 in Table III) mounted on Chamber (#10). |
| | | | Regulates internal solenoid valve which controls |
| | | | flow of 100% CO₂ from CO₂ Supply Tank with |
| | | | Regulator (#16) to CO₂ Supply Fitting (#68 in |
| | | | Table III). |
| 15 | Temperature | Omega | Model CN76000. Input from RTD (#60) |
| | Controller | | |
| 16 | CO₂ Supply | Matheson (Tank); | Supplies Chamber (#10) with 100% CO₂ through |
| | Tank with | Regulator (Fisher) | CO₂ Controller (#14). |
| | Regulator | | |
| 17 | Temperature | Omega | Model CN76000. Input from RTD (#59) |
| | Controller | | |
| 18 | Motorized Stage | Ludl | X-Y stage with 4.5″ × 3.25″ travel. Mounts on |
| | | | Inverted Microscope (#20); motion controlled by |
| | | | 2 each Microstepper Motor Controller Boards |
| | | | 73000500 and Microstepper Power Boards |
| | | | 73000503 installed in Microscope Controller |
| | | | (#28). |
| 20 | Inverted | Nikon | Diaphot 300, equipped with 100 white light, |
| | Microscope | | ELWD condenser, 6-place nosepiece with 4x and |
| | | | 10x phase objectives and 20x and 40x ELWD |
| | | | phase objectives, HMX-4 lamphouse with Hg |
| | | | bulb, and epifluorescence attachment. Mounts |
| | | | Motorized Stage (#18), Motorized Focus Drive |
| | | | Assembly (#22), High-Speed Shutter for |
| | | | Transmitted Light (#24), High-Speed Dual Filter |
| | | | Wheel with Shutter for Fluorescence (#26), and |
| | | | Video-Rate (#32) and Cooled (#34) CCD |
| | | | Cameras |
| 22 | Motorized Focus | Ludl | Model 73000901 Focus Drive Motor Assembly |
| | Drive Assembly | | and Model 99A006 Z-axis Control Card. Focus |
| | and Controller | | Drive Motor Assembly mounts on focus control |
| | | | of Inverted Microscope (#20) and controls focus |
| | | | through action of Control Card installed in |
| | | | Microscope Controller (#28). |
| 24 | High-Speed | Ludl | Model 99A043 shutter with microscope adapter |
| | Shutter for | | flange mounts on Inverted Microscope (#20). |
| | Transmitted | | Position of shutter (i.e., open or close) controlled |
| | Light | | by Model 73000800 board in Microscope |
| | | | Controller (#28). |
| 26 | High-Speed Dual | Ludl | Model 99A076 high-speed dual 6 position filter |
| | Filter Wheel | | wheel with 100 ms switching between filters and |
| | with Shutter for | | high-speed shutter for excitation by |
| | Fluorescence | | epifluorescence. Position of filter wheel and |
| | | | shutter controlled by Model 73000800 board in |
| | | | Microscope Controller (#28). |
| 28 | Microscope | Ludl | Model 990082 19″ automation electronics |
| | Controller | | console with joystick. Controls movement of |
| | | | Motorized Stage (#18) and Motorized Focus |
| | | | Drive Assembly (#22) and position of High- |
| | | | Speed Shutter for Transmitted Light (#24) and |
| | | | High-Speed Dual Filter Wheel with Shutter for |
| | | | Fluorescence (#26) through communications with |
| | | | Quadra 950 (#42) by RS-232 interface. |
| 30 | Joystick | Ludl | Model 73000362. X-Y action controls sets initial |
| | | | position of Motorized Stage (#18); Z-axis digipot |
| | | | set initial position of Motorized Focus Drive |
| | | | Assembly (#22). |
| 32 | Cooled CCD | Photometrics | High performance cooled CCD camera with |
| | Camera | | Kodak Model KAF1400 Grads 1 chip with 1317 × 1035 |
| | | | pixel resolution, and 12-bit/pixel gray |
| | | | scale resolution at 500 kHz and CE200A Camera |
| | | | Electronics Unit controller. Output to PC (#42). |
| 38 | Imaging Board | Photometrics | Photometrics, PCI interface board for KAF 1400 |
| | | | camera. |
| 42 | Pentium III PC | Gateway | Pentium III PC with 256 MB RAM, 20GB |
| | | | harddisk, connected to a Cooled CCD Camera. |
| 44 | Video Board | Gateway, Inc | AccelGraphics Permedia 2 AGP 8 MB Video |
| | | | Card |
| 46 | Computer | Gateway | 17″ Multiscan color monitor. Input from PC |
| | Monitor | | |
| 66 | CO2 Sensor | Valtronics | Valtronics, 3463 Double Springs Road, Valley |
| | | | Springs CA 95252 model 2007DHH-R, 0-10% |
| | | | CO2 |
| 68 | Supply Fitting | McMaster-Carr | McMaster-Carr Part # 52065K113 ⅛ T × ⅛ NPT |
| 72 | Quick | McMaster-Carr | McMaster-Carr Part # 52065K 151 ⅛ T × ⅛ T |
| | Disconnect | | |
| | Fitting | | |
| 108 | Syringe | Kloehn Ltd | Kloehn Part # 50300 |
| 114 | Distribution | Kloehn Ltd | Kloehn Part # 50120 |
| | Valve | | |

TABLE III
Components of Chamber for Automated Single-Cell Culture System (Component #10 in FIG. 1 and Table II)

| Component # | Name | Description |
|---|---|---|
| 50 | Chamber Body | Constructed of anodized aluminum. Forms enclosed chamber (#10 in Table II) |
| | | by assembly with Chamber Cover (#52) and Turbine Housing (#76). Mounts |
| | | screwed in Thermocouple Fitting (#60) with Thermocouple (#58), 2 Heating |
| | | Cartridges (#62) secured with Heating Cartridge Retaining Screws (#64), CO₂ |
| | | Sensor (#66) by two 1½″ × 3/16″ hex-nut headed screws, screwed-in CO₂ |
| | | Supply Fitting (#68), screwed-in Pressure Relief Fitting (#70), and 3 screwed |
| | | in Unused Port Plugs (#74). Gas-tight seal between Chamber Body and |
| | | Chamber Cover (#52) maintained by tightening 8 0.50″ × 0.19″ hex-nut |
| | | headed screws with Chamber Cover Gasket (#56) in place; gas tight seal |
| | | between Chamber Body and Turbine Housing maintained by tightening two |
| | | 1¼″ × 3/16″ hex-nut headed screws with Turbine Housing O-Ring (#86) in |
| | | place. |
| 52 | Chamber Cover | Constructed of anodized aluminum. Glass Observation Window (#54) glued |
| | | with silicone rubber into inset. Mounted on top of Chamber Body (#50) of |
| | | chamber by 8 0.50″ × 0.19″ hex-nut headed screws. Gas tight seal between |
| | | Chamber Body and Chamber Cover maintained by tightening screws with |
| | | Chamber Cover Gasket (#56) in place. |
| 54 | Glass | One each 5.00″ × 3.41″ × 0.01″ optical-grade glass slides glued by silicone |
| | Observation | rubber into inset on bottom of Chamber Body (#50) and inset on top of |
| | Windows (2) | Chamber Cover (#52). |
| 56 | Chamber Cover | Silicone rubber O-ring gasket (size #162) forms gas-tight seal between |
| | Gasket | Chamber Body (#50) and Chamber Cover (#52) with tightening of 8 0.50″ × 0.19″ |
| | | hex-nut headed screws. Outer dimensions 6.30″ × 4.33″, inner |
| | | dimensions 5.25″ × 3.50″, thickness 0.01″. |
| 58 | RTD (Resistance | RDF Corp Part # 29228-Tol-B-24 |
| | Temperature | |
| | Device) | |
| 62 | Heating | 20 watt McMaster-Carr heating cartridge. Each mounts into ports on front of |
| | Cartridges (2) | Chamber Body (#50) and secured in place by a Heating Cartridge Retaining |
| | | Screw (#64). Each connected by insulated electrical wire to Temperature |
| | | Controller (#12 in Table II). |
| 64 | Heating | One each secures on Heating Cartridge (#62) in sidewalls of Chamber Body |
| | Cartridge | (#50) through ports on front of Chamber Body. Constructed of anodized |
| | Retaining Screws | aluminum. Mount by screwing into Chamber Body. |
| | (2) | |
| 68 | CO₂ Supply | Teflon elbow, ⅛ NPT, screwed and sealed with teflon tape into front port on |
| | Fitting | Chamber Body (#50). Connected by Tygon tubing to CO₂ Controller (#14 in |
| | | Table II). |
| 74 | Unused Port | Stainless steel fittings with threads wrapped in Teflon tape and screwed into |
| | Plugs (3) | unused ports of Chamber Body (#50). |
| 90 | House Air | Teflon elbow, ⅛ NPT, screwed and sealed with teflon tape into side port on |
| | Fittings (2) | Turbine Housing (#76). Connected by Tygon tubing to House air supply. |
| 230 | Advanced Liquid | Advanced Liquid Handling model MBP 2000 (Williams Bay, WI) |
| | Handling | |

What is claimed is:

1. A protein-analysis method comprising:
(A) bringing a protein into contact with at least a first disease-model cell and a second disease-model cell, respectively, wherein each of said first and second cells is located in a separate well; then
(B) determining the dynamic state of each of said cells, whereby a data set is generated for each cell; and
(C) analyzing the data set for said first cell and the data set for said second cell, to obtain information about the function of said protein.

2. A method according to claim 1, wherein said first disease-model cell and said second disease-model cell relate to the same disease model.

3. A method according claim 1, wherein the data sets of step (C) address a plurality of cell parameters.

4. A method according to claim 1, wherein said determination of the dynamic state comprises imaging each of said cells by either visible or fluorescent light, or both.

5. A method according to claim 1, wherein step (A) comprises bringing said protein into contact with a first plurality of first disease-model cells and a second plurality of second disease-model cells, respectively, and wherein said information distinguishes a subpopulation of at least one of said first and second pluralities.

6. A method according to claim 1, further comprising providing a plurality of proteins, wherein step (A) comprises bringing into contact, with N number of disease-model cells, a chosen protein from said plurality such that each of at least some of said N cells contacts a different protein from said plurality, N being an integer greater than 2.

7. A method according to claim 6, wherein more than one well receives the same protein from said plurality of proteins.

8. A method according to claim 6, wherein at least one well does not receive a protein from said plurality of proteins.

9. A method according to claim 6, wherein at least one well receives more than one protein from said plurality of proteins.

10. A method according to claim 6, wherein the data sets of step (C) address a plurality of M cell parameters, M being an integer of 1 or greater.

11. A method according to claim 10, wherein said cell parameters comprise two or more of the measured parameters enumerated in Table I.

12. A method according to claim 10, wherein a data set of step (C) is organized as an N×M array of values.

13. A method according to claim 10, wherein either said first disease-model cell or said second disease-model cell employs an oncogenesis disease model.

14. A method according to claim 10, wherein either said first disease-model cell or said second disease-model cell employs a primary immune response disease model.

15. A method according to claim 10, wherein either said first disease-model cell or said second disease-model cell employs an angiogenesis disease model.

16. An integral array of biochambers, each (i) comprising a well in which a disease-model cell is located and (ii) defining a separate, closed environment for said cell, wherein each well contains a protein and said array presents a predetermined pattern of association between wells and proteins.

17. A protein-analysis method comprising:
(A) disposing a first disease-model cell in a first well in a manner wherein at least one cell is individually observable;
(B) disposing a second disease-model cell in a second well in a manner wherein at least one cell is individually observable;
(C) bringing a protein into contact with said first and second disease-model cells;
(D) repeatedly observing the first and second disease-model cells;
(E) compiling data in the form of data sets pertaining to a change in at least one of a plurality of observable characteristics of each of the respective first and second disease-model cells, prior to and subsequent to the protein being contacted with the first and second disease-model cells; and
(F) analyzing the data sets to obtain information about the function of the protein.

18. A method according to claim 17, wherein said first disease-model cell and said second disease-model cell relate to the same disease model.

19. A method according to claim 17, further comprising adding a modifying agent.

20. A method according to claim 17, wherein steps (A) through (D) are implemented robotically, within a closed environment.

21. A method according to claim 17, wherein steps (A) through (F) are implemented robotically.

22. A method according to claim 17, wherein the step of repeatedly observing is carried out optically.

23. A method according to claim 17, wherein the observable characteristics are selected from the group consisting of cell movement, cell division, apoptosis, morphology, adherence and physiological function

24. A method according to claim 17, wherein the observable characteristics comprise the measured parameters enumerated in Table I.

25. A method according to claim 17, further comprising means for selectively adding a modifying agent in addition to the protein.

26. A protein-analysis apparatus comprising:
means for disposing a plurality of first disease-model cells in a first well in a manner wherein at least one of the first disease-model cells is individually observable;
means for disposing a plurality of second disease-model cells in a second well a manner wherein at least one of the second disease-model cells is individually observable;
means for bringing a protein into contact with at least one of the first and second disease-model cells;
means for repeatedly observing the first and second disease-model cells;
means for compiling and analyzing data in the form of data sets that pertain to a change in at least one of a plurality of observable characteristics of each of the respective first and second disease-model cells, prior to and subsequent to the protein being contacted with the first and second disease-model cells.

27. A protein-analysis method comprising:
(A) disposing a disease-model cell in a well in a manner wherein at least one cell is individually observable;
(B) bringing a plurality of proteins into contact with said disease-model cell;
(D) repeatedly observing said disease-model cell;
(E) compiling data in the form of data sets pertaining to a change in at least one of a plurality of observable characteristics of disease-model cell, prior to and subsequent to the proteins being contacted with the disease-model cell; and
(F) analyzing the data sets to obtain information about the function of the proteins.

28. The method of claim 27, further comprising isolating a protein of interest by splitting said plurality into a smaller number of pluralites and repeating steps (A) thru (F), using said smaller number of pluralites for step (13).

29. The method of claim 27, further comprising isolating a protein of interest by splitting said plurality into individual proteins and repeating steps (A) thru (F) for each of said proteins.
